(19) United States
(12) Patent Application Publication
(10) Pub. No.: US 2006/0057106 A1
(43) Pub. Date: Mar. 16, 2006

(54) Freeze-dried interferon-y composition for transpulmonary administration and inhalation system therefor

(76) Inventors: Chikamasa Yamashita, Tokushima (JP); Shigeru Ibaragi, Tokushima (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/538,781

(22) PCT Filed: Dec. 12, 2003
(86) PCT No.: PCT/JP03/15957

§ 371 Date: Jun. 10, 2005

(30) Foreign Application Priority Data
Dec. 13, 2002 (JP) 2002-363026

(51) Int. Cl. A61K 38/21

(52) U.S. Cl. 424/85.6

(57) ABSTRACT

The present invention provides a freeze-dried interferon-γ composition for transpulmonary administration which can maintain IFN-γ stably, and can be prepared into fine particles in a vessel at the time of use. A freeze-dried interferon-γ composition for transpulmonary administration of the present invention has the following properties (i) to (iv): (i) containing at least one hydrophobic stabilizer selected from the group consisting of hydrophobic amino acids, dipeptides of hydrophobic amino acids, tripeptides of hydrophobic amino acids and derivatives of hydrophobic amino acids and salts thereof; at least one hydrophilic stabilizer selected from the group consisting of hydrophilic amino acids, dipeptides of hydrophilic amino acids, tripeptides of hydrophilic amino acids, derivatives of hydrophilic amino acids and salts thereof; and interferon-γ(ii) a non-powder cake-like form; (iii) a disintegration index of 0.015 or more; and (iv) becoming fine particles having a mean particle diameter of 10 microns or less or a fine particle fraction of 10% or more upon receipt of an air impact having an air speed of at least 1 m/sec and an air flow rate of at least 17 ml/sec.

### TECHNICAL FIELD

The present invention relates to a freeze-dried composition containing interferon-γ for transpulmonary administration. More specifically, the present invention relates to a freeze-dried interferon-γ composition which can stably maintain interferon-γ and can be made into fine particle powder suitable for transpulmonary administration (hereinafter, referred to as dry powdered interferon-γ preparation for transpulmonary administration) at the time of use.

The present invention relates to a dry powder interferon-γ inhalation system for transpulmonary administration employing the freeze-dried composition. More specifically, the present invention relates to a dry powder inhalation system for transpulmonary administration according to which the freeze-dried composition provided housed in a vessel can be prepared into a form suitable for transpulmonary administration by being made into fine particles at the time of use, and administered by inhalation as is.

Furthermore, the present invention encompasses the following inventions related to the dry powder interferon-γ inhalation system for transpulmonary administration. Specific examples of these inventions include a method for manufacturing dry powdered interferon-γ preparation for transpulmonary administration from the freeze-dried interferon-γ composition, a method for transpulmonary administration by inhalation using the freeze-dried interferon-γ composition and use of the freeze-dried interferon-γ composition for preparing a dry powdered interferon-γ preparation for transpulmonary administration at the time of use. Hereinafter, in this specification, the term “fine particles” includes particle powder. Forms of the fine particles are not particularly limited.

### BACKGROUND ART

In general, with regard to transpulmonary administration, it is known that the active ingredient contained in a medicine can be delivered into the lungs efficiently by making the mean particle diameter of the active ingredient be 10 microns or less, preferably 5 microns or less. The current situation with conventional inhalations for transpulmonary administration is thus that, to make the medicine have a particle diameter suitable for transpulmonary administration in advance, fine particles are prepared by a spray drying method, a jet milling method or the like, and possibly further processing is carried out, and then the fine particles are provided filled into a dry powder inhaler (for example, International Publication No. WO 95/31479 and International Publication No. WO91/16038).

Moreover, conventional methods for preparing dry powdered inhalation for transpulmonary administration require an operation in which the fine powder prepared is collected from the spray drying apparatus or jet milling apparatus and is subdivided and filled into vessels. It is thus inevitable that, accompanying this operation, problems will arise such as the yield of the preparation decreasing due to collection or filling loss and the cost rising correspondingly, and the preparation being contaminated with impurities. Moreover, in general it is difficult to subdivide and fill the powder in small amounts with good accuracy. If the spray drying method or the freeze drying-jet milling method, for which such subdividing and filling of small amounts in powder form is essential, is used, then it is thus necessary to establish a method of filling with small amounts and good accuracy of powder. In actual fact, details of a system, apparatus and method for filing with a fine powder are disclosed in U.S. Pat. No. 5,826,633.

Interferons are well known as active ingredients capable of being used for transpulmonary administration, and which have biological properties such as antiviral properties, immune modulating properties or cell proliferation suppressing properties. Interferons are proteins and thus inherently prone to lose activity due to heat, pH and the like. In particular, interferon-γ among various types of interferon has disadvantages such that the activity is easily lost and stability is poor. Therefore, a dry powdered inhalation for transpulmonary administration containing interferon-γ as an active ingredient is disadvantaged in that the activity of interferon-γ is reduced during formulation or with time, in addition to the problems of the conventional dry powdered inhalations for transpulmonary administration.

### DISCLOSURE OF THE INVENTION

It is an object of the present invention to solve the various problems of the above-mentioned conventional powdered inhalations for transpulmonary administration. Specifically, it is an object of the present invention to provide a freeze-dried composition for transpulmonary administration which can be made into fine particles in the vessel at the time of usage.

Moreover, it is an object of the present invention to provide a novel preparation system and administration system that enable a freeze-dried interferon-γ composition that has been housed in vessels in advance subdivided into single doses of active ingredient to be made into fine particles down to a particle diameter suitable for transpulmonary administration by inhalation in the vessel at the time of usage, and then be used for transpulmonary administration as is.

The present inventors carried out assiduous studies to attain the above object, and as a result discovered that interferon-γ having the following properties (i) to (iv) can be made into fine particles by a relatively low air impact while still housed in the vessel and interferon-γ in the composition is endowed with excellent stability: (i) at least one hydrophobic stabilizer selected from the group consisting of hydrophobic amino acids, dipeptides of hydrophobic amino acids, tripeptides of hydrophobic amino acids, derivatives of hydrophobic amino acids and salts thereof; at least one hydrophilic stabilizer selected from the group consisting of hydrophilic amino acids, dipeptides of hydrophilic amino acids, tripeptides of hydrophilic amino acids and derivatives of hydrophilic amino acids and salts thereof; and interferon-γ; (ii) having a non-powder cake-like form; (iii) having a disintegration index of at least 0.015; and (iv) upon receiving an air impact having an air speed of at least 1 m/sec and an air flow rate of at least 17 ml/sec becoming fine particles having a mean particle diameter of no more than 10 microns or a fine particle fraction of at least 10%.

The present inventors carried out further studies, and as a result discovered that by using a freeze-dried interferon-γ composition, a single dose of which has been housed in a non-powder form in a vessel, combined with a device comprising means for introducing air at a prescribed speed and flow rate into the vessel so as to be capable of applying a prescribed air impact to the composition, and means for discharging from the vessel the powdered composition that has been made into fine particles, then the freeze-dried preparation can be prepared into a fine particle powder form suitable for transpulmonary administration easily by a user at the time of use (specifically, at the time of inhalation), and the fine particle powder can be administered by inhalation as is.

The present invention was developed based on this knowledge.

(I) The present invention includes the following freeze-dried interferon-γ compositions for transpulmonary administration.

Item 1. A freeze-dried interferon-γ composition for transpulmonary administration having the following properties (i) to (iv):
(i) containing at least one hydrophobic stabilizer selected from the group consisting of hydrophobic amino acids, dipeptides of hydrophobic amino acids, tripeptides of hydrophobic amino acids, derivatives of hydrophobic amino acids and salts thereof; at least one hydrophilic stabilizer selected from the group consisting of hydrophilic amino acids, dipeptides of hydrophilic amino acids, tripeptides of hydrophilic amino acids, derivatives of hydrophilic amino acids and salts thereof; and interferon-γ;
(ii) a non-powder cake-like form;
(iii) a disintegration index of 0.015 or more; and
(iv) becoming fine particles having a mean particle diameter of 10 microns or less or a fine particle fraction of 10% or more upon receiving an air impact having an air speed of at least 1 m/sec and an air flow rate of at least 17 ml/sec.

Item 2. The freeze-dried interferon-γ composition according to Item 1, wherein the hydrophilic stabilizer is at least one selected from the group consisting of basic amino acids, neutral hydroxy amino acids, dipeptides of these amino acids, tripeptides of these amino acids, derivatives of these amino acids and salts thereof.

Item 3. The freeze-dried interferon-γ composition according to Item 1, wherein the hydrophilic stabilizer is at least one selected from the group consisting of basic amino acids, dipeptides of basic amino acids, tripeptides of basic amino acids, derivatives of basic amino acids and salts thereof.

Item 4. The freeze-dried interferon-γ composition according to Item 1, wherein the hydrophilic stabilizer is at least one selected from the group consisting of neutral hydroxy amino acids, dipeptides of neutral hydroxy amino acids, tripeptides of neutral hydroxy amino acids, derivatives of neutral hydroxy amino acids and salts thereof.

Item 5. The freeze-dried interferon-γ composition according to Item 1, wherein the hydrophilic stabilizer is at least one selected from the group consisting of arginine, lysine, histidine, threonine, dipeptides of these amino acids, tripeptides of these amino acids, derivatives of these amino acids and salts thereof.

Item 6. The freeze-dried interferon-γ composition according to Item 1, wherein the hydrophobic stabilizer is at least one selected from the group consisting of hydrophobic amino acids, dipeptides of hydrophobic amino acids, tripeptides of hydrophobic amino acids, derivatives of hydrophobic amino acids and salts thereof.

Item 7. The freeze-dried interferon-γ composition according to Item 1, wherein the hydrophobic stabilizer is at least one selected from the group consisting of valine, leucine, isoleucine, phenylalanine and salts thereof.

Item 8. The freeze-dried interferon-γ composition according to Item 1, wherein the content of the hydrophilic stabilizer is 1 to 500 parts by weight per 100 parts by weight of the hydrophobic stabilizer.

Item 9. The freeze-dried interferon-γ composition according to Item 1, wherein the disintegration index is 0.02 or more.

Item 10. The freeze-dried interferon-γ composition according to Item 1, wherein the disintegration index is from 0.015 to 1.5.

Item 11. The freeze-dried interferon-γ composition according to Item 1, becoming fine particles having a mean particle diameter of 10 microns or less or a fine particle fraction of 10% or more upon receipt of an air impact having an air speed of at least 2 m/sec and an air flow rate of at least 17 ml/sec.

Item 12. The freeze-dried interferon-γ composition according to Item 1, becoming fine particles having a mean particle diameter of 10 microns or less or a fine particle fraction of 10% or more upon receipt of an air impact having an air speed of at least 1 m/sec and an air flow rate of at least 20 ml/sec.

Item 13. The freeze-dried interferon-γ composition according to Item 1, becoming fine particles having a mean particle diameter of 5 microns or less or a fine particle fraction of 20% or more upon receipt of the air impact.

Item 14. The freeze-dried interferon-γ composition for transpulmonary administration according to Item 1, having the following properties (i) to (iv):
(i) containing at least one hydrophobic stabilizer selected from the group consisting of hydrophobic amino acids, dipeptides of hydrophobic amino acids, tripeptides of hydrophobic amino acids, derivatives of hydrophobic amino acids and salts thereof; at least one hydrophilic stabilizer selected from the group consisting of hydrophilic amino acids, dipeptides of hydrophilic amino acids, tripeptides of hydrophilic amino acids, derivatives of hydrophilic amino acids and salts thereof; and interferon-γ;
(ii) a non-powder cake-like form;
(iii) a disintegration index of 0.015 to 1.5; and
(iv) becoming fine particles having a mean particle diameter of 10 microns or less or a fine particle fraction of 10% or more upon receipt of an air impact having an air speed in the range of 1 to 300 m/sec and an air flow rate in the range of 17 ml/sec to 15 L/sec.

(II) The present invention includes the following dry powder interferon-γ inhalation systems for transpulmonary administration.

Item 15. A dry powder interferon-γ inhalation system for transpulmonary administration, using a combination of:
(1) a vessel housing the freeze-dried interferon-γ composition for transpulmonary administration according to any of Items 1 to 14; and
(2) a device comprising means capable of applying an air impact having an air speed of at least 1 m/sec and an air flow rate of at least 17 ml/sec to the freeze-dried composition in said vessel, and means for discharging the powder-form freeze-dried composition that has been made into fine particles.

Item 16. The dry powder interferon-γ inhalation system for transpulmonary administration according to Item 15, wherein the vessel and the device are used in combination at the time of inhalation.

Item 17. The dry powder interferon-γ inhalation system for transpulmonary administration according to Item 15, wherein the device is:
i) a dry powder inhaler for transpulmonary administration, being a device used for making a freeze-dried composition that has been housed in non-powder form in a vessel into fine particles, and administering the resulting fine particles to a user by inhalation,
comprising a needle part having an air jet flow path, a needle part having a discharge flow path, air pressure-feeding means for feeding air into the air jet flow path of said needle part, and an inhalation port that communicates with the discharge flow path of said needle part,
and characterized by being constituted such that a stopper that seals up said vessel is pierced by said needle parts, thus communicating the air jet flow path and the discharge flow path with the inside of said vessel, and air is jetted into said vessel through said air jet flow path using said air pressure-feeding means, thus making said freeze-dried composition into fine particles by the impact of the jetted air, and discharging the fine particles obtained from the inhalation port via said discharge flow path, or
ii) a dry powder inhaler for transpulmonary administration, being a device used for making a freeze-dried composition that has been housed in non-powder form in a vessel into fine particles, and administering the resulting fine particles to a user by inhalation,
comprising a needle part having a suction flow path, a needle part having an air introduction flow path, and an inhalation port that communicates with said suction flow path,
and characterized by being constituted such that, in a state in which a stopper sealing up said vessel has been pierced by said needle parts, through the inhalation pressure of the user, air in said vessel is inhaled from said inhalation port, and at the same time outside air flows into said vessel, at a negative pressure, through said air introduction flow path, and as a result said freeze-dried composition is made into fine particles by the impact of the air flowing in, and the fine particles obtained are discharged from the inhalation port through said suction flow path.

Item 18. The dry powder interferon-γ inhalation system for transpulmonary administration according to Item 17, as the device, using the dry powder inhaler comprising;
a holder part for holding a vessel that is sealed up with a stopper and houses a freeze-dried composition in a non-powder cake-like form that will be made into fine particles upon receiving an air impact,
means for applying an air impact to said freeze-dried composition in said vessel, and sucking said freeze-dried composition in a powder-form that has been made into fine particles by the air impact out from said vessel,
comprising a needle part having a suction flow path for sucking said freeze-dried composition out from said vessel, and an air introduction flow path for introducing outside air into said vessel,
a suction port that communicates with said suction flow path of said needle part,
a guide part for guiding said holder part in the axial direction of said needle part,
a holder operating part that has a mechanism part for, when said vessel is held by said holder part, advancing the vessel towards a needle tip of said needle part to pierce the stopper of the vessel with said needle tip, and retreating the vessel from said needle tip to separate the stopper of the vessel from said needle tip, and an operator that operates the mechanism part, and is constituted such that said operating member can be operated with a force smaller than the force necessary for the mechanism part to pierce the stopper of the vessel with said needle part,
and a housing that supports said needle part and is for providing said suction port, said guide part and said holder operating part,
and constituted such that, in a state in which said stopper has been pierced by said needle part to communicate the suction flow path and the air introduction flow path of said needle part with the inside of said vessel and position the tip of the air introduction flow path at said freeze-dried composition, through the inhalation pressure of a user, air in said vessel is inhaled from said suction port, and air is made to flow into said vessel through the air introduction flow path, thus applying an air impact to the freeze-dried composition in said vessel.

Item 19. The dry powder interferon-γ inhalation system for transpulmonary administration according to Item 15, using a combination of:
(1) a vessel housing the freeze-dried interferon-γ composition for transpulmonary administration according to Item 14; and
(2) a device comprising means capable of applying said air impact to the freeze-dried composition in said vessel, and means for discharging the powder-form freeze-dried composition that has been made into fine particles.

(III) The present invention includes the following methods of manufacturing a dry powdered interferon-γ preparation for transpulmonary administration.

Item 20. A method of manufacturing a dry powdered interferon-γ preparation for transpulmonary administration, comprising:
introducing air into a vessel to apply to a freeze-dried composition containing a single dose of interferon-γ according to any of Items 1 to 14 an air impact having an air speed of at least 1 m/sec and an air flow rate of at least 17 ml/sec using a device capable of applying said air impact to the freeze-dried composition in the vessel,
thereby making said freeze-dried composition into fine particles having a mean particle diameter of 10 microns or less or a fine particle fraction of 10% or more.

Item 21. The method of manufacturing a dry powdered interferon-γ preparation for transpulmonary administration according to Item 20, wherein the fine particles prepared have a mean particle diameter of 5 microns or less or a fine particle fraction of 20% or more.

Item 22. The method of manufacturing a dry powdered interferon-γ preparation for transpulmonary administration according to Item 20, being a method of making the freeze-dried composition into fine particles in a vessel having a volume of 0.2 to 50 ml.

Item 23. The method of manufacturing a dry powdered preparation for transpulmonary administration according to Item 20, carried out by using a device having means capable of applying an air impact having an air speed of at least 2 m/sec and an air flow rate of at least 17 ml/sec to the freeze-dried composition in the vessel, and introducing air having the air impact into the vessel housing the freeze-dried composition.

Item 24. The method of manufacturing a dry powdered preparation for transpulmonary administration according to Item 20, carried out by using a device having means capable of applying an air impact having an air speed in a range of 1 to 300 m/sec and an air flow rate of at least 17 ml/sec to the freeze-dried composition in the vessel, and introducing air having the air impact into the vessel housing the freeze-dried composition.

Item 25. The method of manufacturing a dry powdered preparation for transpulmonary administration according to Item 20, carried out by using a device having means capable of applying an air impact having an air speed of at least 1 m/sec and an air flow rate of at least 20 ml/sec to the freeze-dried composition in the vessel, and introducing air having the air impact into the vessel housing the freeze-dried composition.

Item 26. The method of manufacturing a dry powdered preparation for transpulmonary administration according to Item 20, carried out by using a device having means capable of applying an air impact having an air speed of at least 1 m/sec and an air flow rate in a range of 17 ml/sec to 15 L/sec to the freeze-dried composition in the vessel, and introducing air having the air impact into the vessel housing the freeze-dried composition.

Item 27. The method of manufacturing a dry powdered interferon-γ preparation for transpulmonary administration according to Item 20, comprising:
introducing air into a vessel to apply to a freeze-dried composition containing a single dose of an interferon-γ according to Item 14 an air impact having an air speed of at least 1 m/sec and an air flow rate of at least 17 ml/sec using a device capable of applying said air impact to the freeze-dried composition in the vessel,
thereby making said freeze-dried composition into fine particles having a mean particle diameter of 10 microns or less or a fine particle fraction of 10% or more.

Item 28. The method of manufacturing a dry powdered interferon-γ preparation for transpulmonary administration according to Item 20, comprising making the freeze-dried interferon-γ composition for transpulmonary administration into fine particles by using the dry powder interferon-γ inhalation system for transpulmonary administration according to any of Items 15 to 19.

(IV) Furthermore, the present invention includes the following transpulmonary administration methods characterized by using the freeze-dried interferon-γ composition for transpulmonary administration as described above. According to the transpulmonary administration method, a freeze-dried interferon-γ composition that has been housed in a non-powder form in a vessel is made into a fine particle powder suitable for transpulmonary administration at the time of use so that a user (patient) can administer the fine-particle-form powdered preparation by inhalation.

Item 29. A transpulmonary administration method comprising: making the freeze-dried interferon-γ composition for transpulmonary administration containing a single dose of interferon-γ according to any of Items 1 to 14 into fine particles having a mean particle diameter of 10 microns or less or a fine particle fraction of 10% or more by applying an air impact having an air speed of at least 1 m/sec and an air flow rate of at least 17 ml/sec to the freeze-dried composition at the time of use, and administering the resulting fine particle powder to a user by inhalation.

Item 30. The transpulmonary administration method according to Item 29, wherein the freeze-dried interferon-γ composition for transpulmonary administration is housed in a vessel, and the fine particle powder are made using a device comprising means capable of applying the air impact to the freeze-dried composition in the vessel and means for discharging the resulting fine particle powder-form freeze-dried composition out of the vessel.

Item 31. The transpulmonary administration method according to Item 29, wherein the freeze-dried interferon-γ composition for transpulmonary administration made into fine particles is administered to a user by inhalation by using the dry powder interferon-γ inhalation system for transpulmonary administration according to any of Items 15 to 19.

Item 32. The transpulmonary administration method according to Item 29, wherein the air speed is 1 to 250 m/sec.

Item 33. The transpulmonary administration method according to Item 29, wherein the air flow rate is 20 ml/sec to 10 L/sec.

(V) The present invention includes the following uses of a freeze-dried interferon-γ composition.

Item 34. Use of a freeze-dried composition according to any of Items 1 to 14 for transpulmonary administration by inhalation, wherein the freeze-dried interferon-γ composition for transpulmonary administration is used by being formed into fine particles having a mean particle diameter of 10 microns or less or a fine particle fraction of 10% or more.

Item 35. Use of the freeze-dried interferon-γ composition for transpulmonary administration according to any of Items 1 to 14 for manufacture of an interferon-γ dry powdered preparation for transpulmonary administration by inhalation.

### BRIEF DESCRIPTION OF THE DRAWINGS

FIG. 1 is a sectional view showing a dry powder inhaler (jet type 1) used in the dry powder interferon-γ inhalation system for transpulmonary administration of the present invention disclosed as Embodiment 1. Note that, in the drawing, the arrows indicate the flow of external air (likewise in FIGS. 2 and 3 below).

Moreover, the meanings of the various reference numerals are as follows: 1. vessel, 1a. stopper, 2. freeze-dried composition, 3. air jet flow path, 4. discharge flow path, 5. needle part, 6. inhalation port, 7. air intake member, 8. tubular safety cover, 9. air pressure-feeding means, 10. bellows body, 11. intake valve, 12. intake port, 13. discharge valve, 14. discharge port, 15. connecting port (likewise in FIGS. 2 to 11 below).

FIG. 2 is a sectional view showing a dry powder inhaler (self-inhaling type 1) used in the dry powder interferon-γ inhalation system for transpulmonary administration of the present invention disclosed as Embodiment 2. Moreover, the meanings of the various reference numerals areas follows: 16. suction flow path, 17. air introduction flow path, 18. inhalation port, 19. air intake member (likewise in FIG. 3 below).

FIG. 3 is a sectional view showing a dry powder inhaler (self-inhaling type 2) used in the dry powder interferon-γ inhalation system for transpulmonary administration of the present invention disclosed as Embodiment 3.

FIG. 4 is a perspective view showing a dry powder inhaler (self-inhaling type 3) used in the dry powder interferon-γ inhalation system for transpulmonary administration of the present invention disclosed as Embodiment 4. Moreover, the meanings of the reference numerals are as follows: 21. housing, 22. holder part, 27. lid, 28. window, 32. mouthpiece, 32a. mouthpiece cap, 39. connector (likewise in FIGS. 5 to 13 below).

FIG. 5 is a sectional view of the above-mentioned dry powder inhaler (self-inhaling type 3). Moreover, the meanings of the reference numerals are as follows: 20. housing chamber, 21A. hinge, 23. guide part, 24. holder operating part, 26. housing main body, 29. introduction port, 30. check valve, 31. suction port, 33. partition part, 35. remover, 36. lever, 37. mechanism part, 39. connector, 40. hinge, 41. hinge (likewise in FIGS. 6 to 13 below).

FIG. 6(a) is a sectional view of part of the above-mentioned dry powder inhaler (self-inhaling type 3). FIG. 6(b) is a side view of the needle part of this dry powder inhaler. Moreover, the meanings of the reference numerals are as follows: 16a. tip opening of suction flow path 16, 17a. tip opening of air introduction flow path 17, 34. peripheral wall part, 42. second introduction path, 42a. introduction groove in partition part 33, 42b. introduction groove in peripheral wall part 34, 43. gap, 44. one end of second introduction path 42, 45. other end of second introduction path 42, 46. vent hole, 47. wall (likewise in FIGS. 7 to 13 below).

FIGS. 7 to 10 are sectional views for explaining the operation of the above-mentioned dry powder inhaler (self-inhaling type 3). Reference numeral 25 indicates a removal/insertion port.

FIG. 11 is a perspective view of a dry powder inhaler (self-inhaling type 4), which is another embodiment of the present invention. Reference numeral 48. indicates an operator.

FIGS. 12 and 13 are perspective views of a dry powder inhaler (self-inhaling type 5) of another embodiment of the present invention. Reference numeral 49 indicates an operator.

### BEST MODE FOR CARRYING OUT THE INVENTION

(1) Freeze-Dried Interferon-γ Composition for Transpulmonary Administration

The freeze-dried interferon-γ composition of the present invention (hereinafter, sometimes simply referred to as a freeze-dried composition for transpulmonary administration) is a composition containing interferon-γ, hydrophobic stabilizer and hydrophilic stabilizer.

IFN-γ employed in the present invention is not limited in origin. Such IFN-γ includes natural IFN-γ produced using cell culture technology or recombinant IFN-γ produced using DNA recombinant technology, such as IFN-γ 1a, IFN-γ 1b and IFN-γ disclosed in Japanese Published Patent Nos. 1994-173196, 1997-19295, etc.

In the present invention, hydrophobic stabilizers include hydrophiobic amino acids, dipeptides of hydrophobic amino acids, tripeptides of hydrophobic amino acids, derivatives of hydrophobic amino acids and salts thereof.

In the present invention, hydrophobic amino acids include protein-forming amino acids such as valine, leucine, isoleucine, phenylalanine and the like. Dipeptides of hydrophobic amino acids are dipeptides having at least one hydrophobic amino acid and include leucyl-valine, isoleucyl-valine, isoleucyl-leucine, leucyl-glycine and the like. Tripeptides of hydrophobic amino acids are tripeptides having at least one hydrophobic amino acid and include isoleucyl-leucyl-valine, leucyl-glycyl-glycine, and the like. Derivatives of hydrophobic amino acids include amides of hydrophobic amino acids such as L-leucine amido hydrochloride, L-isoleucyl-β-naphthylamido hydrobromide, L-valine-β-naphthyl amide, and the like. Salts include those with an alkali metal such as sodium or potassium; with an alkaline earth metal such as calcium or magnesium; and addition salts with an inorganic acid such as phosphoric acid, hydrochloric acid or hydrobromic acid; or addition salts with an organic acid such as sulfonic acid.

Hydrophobic stabilizers include preferably valine, leucine, isoleucine and phenylalanine, and salts thereof.

Such hydrophobic stabilizers can be used alone or in combination of two or more.

In the present invention, the hydrophilic stabilizers include hydrophilic amino acids, dipeptides of hydrophilic amino acids, tripeptides of hydrophilic amino acids, derivatives of hydrophilic amino acids, and salts thereof.

The hydrophilic amino acids employed in the present invention may be any amino acids insofar as they have a hydrophilic side chain, whether or not the amino acids are protein forming amino acids. Specific examples of the hydrophilic amino acids include basic amino acids such as arginine, lysine, histidine, etc.; neutral hydroxy amino acids such as serine, threonine, etc.; acidic amino acids such as aspartic acid, glutamic acid, etc.; amide amino acids such as asparagine, glutamine, etc.; and other amino acids such as glycine, alanine, cysteine, tyrosine and the like. Basic amino acids are amino acids having basic side chains. Neutral hydroxy amino acids have hydroxyl groups on the side chains. Dipeptides of hydrophilic amino acids have two of the same or different the hydrophilic amino acids. Tripeptides of hydrophilic amino acids have three of the same or different hydrophilic amino acids. Derivatives of hydrophilic amino acids include amides of the hydrophilic amino acids, etc. Salts include those with an alkali metal such as sodium, potassium, etc.; with an alkaline earth metal such as calcium, magnesium, etc.; and addition salts with an inorganic acid such as phosphoric acid, hydrochloric acid or hydrobromic acid, etc., or with an organic acid such as sulfonic acid. Specific examples include salts of hydrophilic amino acids such as arginine hydrochloride, lysine monohydrochloride, lysine dihydrochloride, histidine hydrochloride, histidine dihydrochloride, etc.

The hydrophilic stabilizer preferably include basic amino acids, neutral hydroxy amino acids, dipeptides of these amino acids, tripepdides of these amino acids, derivatives of these amino acids; basic amino acids, dipeptides of the basic amino acids, tripeptides of the basic amino acids, derivatives of the basic amino acids and salts thereof; neutral hydroxyamino acids, dipeptides of neutral hydroxy amino acids, tripeptides of neutral hydroxy amino acids, derivatives of neutral hydroxy amino acids and salts thereof; arginine, lysine, histidine, threonine, dipeptides of these amino acids, tripeptides of these amino acids, derivatives of these amino acids and salts thereof: arginine, lysine, histidine, threonine and salts thereof; arginine, lysine, histidine and salts thereof: and arginine and salts thereof.

These hydrophilic amino stabilizes can be used alone or in combination of two or more.

The content of IFN-γ of the freeze-dried composition for transpulmonary administration can be set according to a target disease, expected effects and the like. The proportion of IFN-γ may for example be in the range of 0.01 to 99.8 wt % of the composition, preferably in the range of 0.1 to 95 wt %, and more preferably in the range of 0.1 to 90 wt %.

The content of hydrophobic stabilizer of the freeze-dried composition for transpulmonary administration can be set according to the proportion of IFN-γ, the type of the hydrophobic stabilizer to be used, disintegration index of the composition, etc. For example, the proportion of the hydrophobic stabilizer may be in the range of 0.1 to 99.89 wt %, preferably within the range of 1 to 95 wt %, and more preferably 5 to 90 wt %.

The proportion of hydrophilic stabilizer of the freeze-dried composition for transpulmonary administration varies according to the content of IFN-γ, the proportion of the hydrophilic stabilizer, and the type of the hydrophilic stabilizer to be used, and thus cannot be determined uniformly, but may be in the range of 0.1 to 99.89 wt %, preferably within the range of 1 to 90 wt %, more preferably 2 to 80 wt %, and more preferably 5 to 70 wt %.

The proportion of hydrophilic stabilizer to hydrophobic stabilizer contained in the freeze-dried composition for transpulmonary administration should be 1 to 500 parts by weight of hydrophilic stabilizer per 100 parts by weight of the hydrophobic stabilizer, preferably 2 to 400 parts by weight, more preferably 5 to 300 parts by weight, still more preferably 8 to 250, and in particular preferably 10 to 200 parts by weight.

The amount of IFN-γ contained in a unit dose (single dose) of the freeze-dried composition for transpulmonary administration is 10,000 to 50,000,000 IU (International Units), preferably 100,000 to 40,000,000 IU, and more preferably 100,000 to 30,000,000 IU.

The amount of hydrophobic stabilizer contained in a single dose of the freeze-dried composition for transpulmonary administration is in the range of 0.01 to 10 mg, preferably 0.1 to 5 mg, and more preferably 0.2 to 0.5 mg.

The amount of hydrophilic stabilizer contained in a single dose of the freeze-dried composition for transpulmonary administration is in the range of 0.01 to 10 mg, preferably 0.1 to 5 mg, and more preferably 0.1 to 2.5 mg.

As described above, hydrophobic stabilizer and hydrophilic stabilizer are mixed with the freeze-dried composition for transpulmonary administration, and thus, the composition can satisfy the desired disintegration index (described later), and IFN-γ of the composition can be endowed with excellent stability.

In addition to the above ingredients, the freeze-dried composition for transpulmonary administration of the present invention can further include monosaccharides such as glucose; disaccharides such as saccharose, maltose, lactose and trehalose; sugar alcohols such as mannitol; oligosaccharides such as cyclodextrin; polysaccharides such as dextran 40 and pullulan; polyhydric alcohols such as polyethylene glycol; fatty acid sodium salts such as sodium caprate; human serum albumin; inorganic salts; surfactants; buffering agents and so on, as long as the end products satisfy the above-mentioned disintegration index. A wide range of surfactants can be used, regardless of whether they are anionic surfactants, cationic surfactants or nonionic surfactants, provided that they are surfactants that are generally used in medicines. Preferable examples are nonionic surfactants such as sorbitan trioleate and polyoxyethylene sorbitan fatty acid esters (for example Tween type surfactants).

The freeze-dried composition for transpulmonary administration of the present invention is a freeze-dried composition having a non-powder cake-like form. In the present invention, ‘non-powder cake-like form freeze-dried composition’ means a dry solid obtained by freeze-drying a solution, and is generally called a ‘freeze-dried cake’. However, even if cracks appear in the cake, the cake breaks into a plurality of large lumps, or part of the cake breaks into a powder during the freeze-drying processor during subsequent handling, this cake is still included as a non-powder-form freeze-dried composition that is the subject of the present invention, provided the effects of the present invention are not impaired.

The freeze-dried composition for transpulmonary administration of the present invention has a disintegration index of at least 0.015. Note that the disintegration index in the present invention is a value characteristic of the freeze-dried composition that can be obtained by measuring following the undermentioned method.

<Disintegration Index>

0.2 to 0.5 ml of a mixture containing target components that will constitute the freeze-dried composition is filled into a vessel having a trunk diameter of 18 mm or 23 mm, and freeze-drying is carried out. Next, 1.0 ml of n-hexane is instilled gently down the wall of the vessel onto the non-powder-form freeze-dried composition obtained. Agitation is carried out for about 10 seconds at 3000 rpm, and then the mixture is put into a UV cell of optical path length 1 mm and optical path width 10 mm, and the turbidity is measured immediately at a measurement wavelength of 500 nm using a spectrophotometer. The turbidity obtained is divided by the total amount (weight) of the components constituting the freeze-dried composition, and the value obtained is defined as the disintegration index.

Here, an example of the lower limit of the disintegration index of the freeze-dried composition of the invention can be given as the above-mentioned 0.015, preferably 0.02, more preferably 0.03, yet more preferably 0.04, and still more preferably 0.05. In particular, 0.1 or 0.15 is preferable. Moreover, there is no particular limitation on the upper limit of the disintegration index of the freeze-dried composition of the invention, but an example can be given as 1.5, preferably 1, more preferably 0.9, yet more preferably 0.8, still more preferably 0.7. The freeze-dried composition of the present invention preferably has a disintegration index in a range constituted from a lower limit and an upper limit selected as appropriate from the above, with the proviso that the disintegration index is at least 0.015. Specific examples of the range of the disintegration index are 0.015 to 1.5, 0.02 to 1.0, 0.03 to 0.9, 0.04 to 0.8, 0.05 to 0.7, 0.1 to 0.7, 0.15 to 1.5, 0.15 to 1.0, and 0.15 to 0.7.

The freeze-dried IFN-γ composition of the present invention has further following properties of a disintegration index in the above-described range and a non-powder cake-like form and becoming fine particles having a mean particle diameter of 10 microns or less or a fine particle fraction of 10% or more upon receipt of an air impact having an air speed of at least 1 m/sec and an air flow rate of at least 17 ml/sec on the basis of properties peculiar to the freeze-dried composition represented by the disintegration index.

As used herein, the mean particle diameter of fine particles indicates a mean particle diameter usually adopted in the industry relating to inhalants for transpulmonary administration. Specifically, the mean particle diameter is not a geometric particle diameter, but an aerodynamic mean particle diameter (mass median aerodynamic diameter, MMAD). The aerodynamic mean particle diameter can be measured by a conventional method. For example, the mass median aerodynamic diameter can be measured using a dry particle size distribution meter fitted with an Aerobreather, which is an artificial lung model (made by Amherst Process Instrument, Inc., USA), a twin impinger (G. W. Hallworth and D. G. Westmoreland: J. Pharm. Pharmacol., 39, 966-972 (1987), U.S. Pat. No. 6,153,224), a multi-stage liquid impinger, a Marple-Miller impactor, an Andersen cascade impactor or the like. Moreover, B. Olsson et al. have reported that delivery of the particles into the lungs increases at the proportion of particles having a mass median aerodynamic diameter of 5 μm or less increases (B. Olsson et al.: Respiratory Drug Delivery V, 273-281(1996)). The fine particle fraction, fine particle dose or the like as measured by a twin impinger, a multi-stage liquid impinger, a Marple-Miller impactor, an Andersen cascade impactor or the like acts as a method of estimating the amount that can be delivered into the lungs.

A preferable freeze-dried composition for transpulmonary administration is one such that, upon receipt of an air impact having an air speed of at least 1 m/sec and an air flow rate of at least 17 ml/sec, the mean particle diameter becomes 10 microns or less and preferably 5 microns or less or a fine particle fraction of 10% or more, preferably 20% or more, more preferably 25% or more, still more preferably 30% or more, and especially more preferably 35% or more.

As described above, the air impact applied to a freeze-dried composition is not limited, as long as it is generated by air having an air speed of at least 1 m/sec and an air flow rate of at least 17 ml/sec. Specific examples of an air impact include an impact generated by an air having a speed of 1 m/sec or more, preferably 2 m/sec or more, more preferably 5 m/sec or more and a still more preferably 10 m/sec or more. Here, there is no limitation on the upper limit of the air speed, but it is generally 300 m/sec, preferably 250 m/sec, more preferably 200 m/sec and yet more preferably 150 m/sec. The air speed is not limited as long as it is arbitrary selected from the range extending from a lower limit to an upper limit; however, the ranges of 1 to 300 m/sec, 1 to 250 m/sec, 2 to 250 m/sec, 5 to 250 m/sec, 5 to 200 m/sec, 10 to 200 m/sec or 10 to 150 m/sec can be given as examples.

Examples of the air impact include those generated by air having an air flow rate of generally 17 ml/sec or more, preferably 20 ml/sec or more and more preferably 25 ml/sec or more. There is no limitation on the upper limit of the air flow rate; however, the air flow rate is generally 900 L/min, preferably 15 L/sec, more preferably 5 L/sec and yet more preferably 4 L/sec. Especially, 3 L/sec is very preferable. More specifically, the air flow rate is not limited as long as it is selected from the range extending from a lower limit to an upper limit; however, examples of such a range include 17 ml/sec to 15 L/sec, 20 ml/sec to 10 L/sec, 20 ml/sec to 5 L/sec, 20 ml/sec to 4 L/sec, 20 ml/sec to 3 L/sec and 25 ml/sec to 3 L/sec.

The freeze-dried composition for transpulmonary administration of the present invention is manufactured by preparing a solution containing IFN-γ, hydrophobic stabilizer and hydrophilic stabilizer, filling the solution of an amount corresponding to a unit dose (single dose) or a plurality of doses into the vessel, and freeze-drying the same. Such a freeze-dried composition for transpulmonary administration can be manufactured by standard freeze-drying methods commonly used in preparing freeze-dried preparations (freeze-dried compositions), such as an injection preparation which is dissolved at the time of usage.

The freeze-dried composition having a non-powder cake-like form containing various active ingredients such as proteins, peptides, polypeptides, genes, nucleic acids, low-molecular-weight drugs; and carriers such as amino acids, sugars, etc., if required, can be made into fine particles having a smaller mean particle diameter or a higher proportion of effective particles (fine particle fraction) upon receipt of an air impact having an air speed of at least 1 m/sec and an air flow rate of at least 17 ml/sec when the amount of salts included in the composition is small. Thus, it is preferable to reduce the concentration of salts contained in the solution used for the freeze-drying process, thereby preparing a freeze-dried composition for transpulmonary administration which can be made into fine particles such that the mean particle diameter can be reduced and the effective particles (fine particle fraction) can be increased upon receipt of an air impact. For example, when salts are contained as preservatives or stabilizers in the refined powder of the active ingredients used or solution, the concentration of the salts contained in the solution used for the freeze-drying process can be reduced either by prior desalination of the refined powder of the active ingredients used or solution or by desalinating the solution itself used for the freeze-drying process. The desalinating method is not limited, but includes ultrafiltration, precipitation, ion-exchange, dialysis under reduced pressure, etc.

To prepare a freeze-dried composition for transpulmonary administration that can be made into fine particles so that the mean particle diameter can be further reduced, and the proportion of effective particles (fine particle fraction) can be further increased upon receipt of an air impact, a small amount of ethanol may be added to the solution used for the freeze-drying process or appropriately set conditions so that granules do not enlarge at freeze-drying.

In the process of manufacturing the freeze-drying composition of the present invention, the freeze-dried composition for transpulmonary administration is prepared so that, for example, a single dose of IFN-γ is included in the vessel, whereby the composition as is can be made into fine particles having a particle diameter suitable for transpulmonary administration in the vessel immediately before transpulmonary administration, and then the powdered composition can be inhaled as is (transpulmonary administration) from the vessel.

The amount of a single dose of the freeze-dried composition for transpulmonary administration of the present invention can be set according to the target disease, expected effects, types of IFN-γ contained, etc. For example, the amount of the single dose may be 0.1 to 20 mg, preferably 0.2 to 15 mg, more preferably 0.3 to 10 mg, still more preferable 0.4 to 8 mg, and in particular preferably 0.5 to 5 mg.

The freeze-dried composition for transpulmonary administration thus obtained can be prepared into fine particles suitable for transpulmonary administration by an air impact having an air speed of at least 1 m/sec and an air flow rate of at least 17 ml/sec. A device for inhaling (transpulmonary administration) the powdered composition includes a dry powder inhaler provided with a means capable of applying an air impact having an air speed of at least 1 m/sec and an air flow rate of at least 17 ml/sec to the freeze-dried composition in the vessel and a means for discharging the powder-form freeze-dried composition having fine particles. Therefore, the above-described device is combined with the vessel housing the freeze-dried composition for transpulmonary administration containing a single dose of IFN-γ, whereby the freeze-dried composition which has been provided in a non-powder form into a powdered preparation comprising fine particles having a mean particle diameter of 10 microns or less or a fine particle fraction of 10% or more, which is a preparation suitable for transpulmonary administration, can be prepared by a user himself/herself at the time of use (the time of inhalation), and administer (take) the powdered preparation.

(II) Dry Powder Interferon-γ Inhalation System for Transpulmonary Administration

The dry powder interferon-γ inhalation system for transpulmonary administration of the present invention comprises a vessel housing the freeze-dried interferon-γ composition for transpulmonary administration and a dry powder inhaler capable of applying the above-described air impact to the freeze-dried composition in the vessel and discharging the produced fine particles.

Hereinbelow, the dry powder inhaler used for the dry powder interferon-γ inhalation system for transpulmonary administration will be described.

The dry powder inhaler used in the present invention can allow both breaking down of the freeze-dried composition into fine particles and administration of the powdered composition to a user by inhalation by comprising \textcircled{1} means capable of applying an air impact to the freeze-dried composition in a degree such that the freeze-dried composition can be made into fine particles, and \textcircled{2} means capable of administering to a user by inhalation the powder-form freeze-dried composition that has been made into fine particles. Note that means \textcircled{1} can be also appreciated as means for introducing air having the above-mentioned air impact into the vessel housing the freeze-dried composition. Moreover, means \textcircled{2} can be also appreciated as means for discharging out of the vessel the powdered preparation that has been made into fine particles in the vessel. In a dry powder inhalation system of the present invention, as long as the device comprises these means, either a conventional publicly-known device or a device which will be developed in the future can also be used.

Specifically, the means \textcircled{1} can be realized by introducing air capable of applying an air impact as above into the vessel housing the freeze-dried composition. Note that the means \textcircled{1} can be altered into means capable of applying an air impact having an air speed of at least 1 m/sec and an air flow rate of at least 17 ml/sec to the freeze-dried composition in the vessel. By using the means \textcircled{2} or via this means, the dry powdered preparation, which has been prepared into a form suitable for transpulmonary administration, can be administered by inhalation to the user such as patient. Note that, for example a chamber or a flow path such that the composition is made into fine particles or scattered may be further provided in the means \textcircled{2}.

The device in question encompasses jet type dry powder inhalers as in (a) below and self-inhaling type dry powder inhalers as in (b) below.

(a) Jet Type Dry Powder Inhaler: Active Powder Inhaler

(a-1) A dry powder inhaler used in the making into fine particles and inhalation of a freeze-dried composition that has been housed in a non-powder form in a vessel,
comprising a needle part having an air jet flow path, a needle part having a discharge flow path, air pressure-feeding means for feeding air into the air jet flow path of the needle part, and an inhalation port that communicates with the discharge flow path,
and being constituted such that a stopper that seals up the vessel is pierced by the needle parts, thus communicating the air jet flow path and the discharge flow path with the inside of the vessel, and air is jetted into the vessel from the air jet flow path using the air pressure-feeding means, thus breaking down the freeze-dried composition into fine particles by the impact of the jetted air, and discharging the fine particles obtained out from the inhalation port via the discharge flow path.

(a-2) The dry powder inhaler described in (a-1) above, being constituted such that the air pressure-feeding means is manually operated and comprises a bellows body having an intake port equipped with an intake valve and a discharge port equipped with a discharge valve, and by contracting the bellows body and thus opening the discharge valve in a state in which the intake valve is closed, air in the bellows body is pressure-fed into the vessel through the air jet flow path of the needle part which communicates with the discharge port, and by expanding the bellows body through an elastic restoring force in a state in which the discharge valve is closed and the intake valve is open, air is introduced into the bellows body.

(a-3) The dry powder inhaler described in (a-1) or (a-2) above, in which the air jet flow path and the discharge flow path are formed in a single needle part.

(b) Self-Inhaling Type Dry Powder Inhaler: Passive Powder Inhaler

(b-1) A dry powder inhaler used for inhaling fine particles obtained by breaking down a freeze-dried composition that has been housed in a non-powder form in a vessel,
comprising a needle part having a suction flow path, a needle part having an air introduction flow path, and an inhalation port that communicates with the suction flow path,
and being constituted such that, in a state in which a stopper that seals up the vessel has been pierced by the needle parts, through the inhalation pressure of a user, air in the vessel is inhaled from the inhalation port, and at the same time outside air flows into the vessel, which is now at a negative pressure, through the air introduction flow path, and as a result the freeze-dried composition is broken down into fine particles by the impact of the air flowing in, and the fine particles obtained are discharged from the inhalation port through the suction flow path.

(b-2) The dry powder inhaler described in (b-1) above, being constituted such that most part of the freeze-dried composition is made into fine particles and discharged from the inhalation port through one inhalation of the user.

(b-3) The dry powder inhaler described in (b-1) or (b-2) above, in which the suction flow path and the air introduction flow path are formed in a single needle part.

The means for introducing air into the vessel (means \textcircled{1} mentioned above) may be means for introducing air from the outside at normal pressure. It is not necessary to use compressed air from a jet mill or the like. There are no limitations on the means for introducing air from the outside. For example, in the case where the jet type dry powder inhaler (active powder inhaler) described above is used, means for artificially introducing external air into the vessel by jetting can be employed. In the case where the self-inhaling type dry powder inhaler (passive powder inhaler) is used, means for naturally introducing outside air into the vessel by suction through negative pressure formed in the vessel when the user inhales can be employed. Moreover, in the former case, i.e. in the jet type dry powder inhaler (active powder inhaler), the method of introducing external air into the vessel by jetting artificially may be manual or may be a method that is carried out automatically using a machine.

The dry powder inhaler of the invention, regardless of the type of the inhaler, whether it is an active powder inhaler or a passive powder inhaler, is capable of breaking down the freeze-dried composition that has been stored in non-powder form in the vessel into fine particles using an impact (jet pressure) of external air introduced into (flowing into) the vessel by the air introduction means.

For example, a vessel, used for freeze-drying can be used here, with no limitations on the material, shape etc. As the material, a plastic mainly including a polyolefin such as polyethylene, polypropylene or polystyrene, glass, aluminum and the like can be given as examples. Moreover, as the shape, a circular cylinder, a cup shape, and a polygonal prism (polygonal pyramid) such as a triangular prism (triangular pyramid), a square prism (square pyramid), a hexagonal prism (hexagonal pyramid) or an octagonal prism (octagonal pyramid) can be given as examples.

To obtain the effects efficiently, the volume of the vessel housing the freeze-dried composition is in a range of 0.2 to 50 ml, preferably 0.2 to 25 ml and more preferably 1 to 15 ml. Moreover, it is desirable to be used the trunk diameter of the vessel be 2 to 100 mm, preferably 2 to 75 mm, more preferably 2 to 50 mm.

Moreover, the amount of the freeze-dried interferon-γ composition for transpulmonary administration housed in the vessel is preferably an amount containing a unit dose (single dose) or a plurality of doses, specifically 2 to 3 doses, of the active ingredient. More preferably, it is an amount containing a unit dose (single dose) of interferon-γ.

Moreover, the air impact generated by the outside air introduced into the vessel is stipulated through the air flow rate at which air flows into the vessel through at least one or a plurality of inhalations of a person or the air speed thus generated. There is no particular limitation on introducing external air with an air flow rate or air speed greater than this, except of course that the durability of the vessel is a limitation. Generally the air flow rate for one inhalation of a person is 5 to 300 L/min, more specifically 10 to 200 L/min. Moreover, in the case of a jet type dry powder inhaler, a device can be used such that the amount of air jetted each time is 5 to 100 ml, preferably 10 to 50 ml. Preferably, adjustment can be carried out such that an air impact generated through an air speed of at least 1 m/sec is applied to the surface of the freeze-dried composition filled in the vessel. A more preferable air impact is an impact generated by an air speed of at least 2 m/sec, a yet more preferable one is an impact generated by an air speed of at least 5 m/sec, and a still more preferable one is an impact generated by an air speed of at least 10 m/sec. Here, there is no particular limitation on the upper limit of the air impact, but an impact generated by an air speed of 300 m/sec can be given as an example. The upper limit is preferably an impact generated through an air speed 250 m/sec, more preferably an impact generated through an air speed 200 m/sec, yet more preferably an impact generated through an air speed 150 m/sec.

There is no particular limitation on the air impact as long as it is generated by air having an air speed arbitrarily selected from the range extending from a lower limit to an upper limit. Specific examples are impacts generated through an air speed in a range of 1 to 300 m/sec, 1 to 250 m/sec, 2 to 250 m/sec, 5 to 250 m/sec, 5 to 200 m/sec, 10 to 200 m/sec or 10 to 150 m/sec.

Here, the speed of the air applied to the freeze-dried composition can be measured as follows. That is, with the jet type dry powder inhaler shown later as Embodiment 1, a mechanism is adopted in which air stored in a bellows body 10 is forcibly introduced onto the freeze-dried composition (cake-like freeze-dried composition: hereinafter also referred to as ‘freeze-dried cake’) that has been filled into the vessel from an air jet flow path 3, thus applying an air impact, and discharging the resulting fine particles from a discharge flow path 4. In this case, the flow rate of the air flowing through the air jet flow path 3 can be calculated by dividing the amount of air stored in the bellows body 10 by the time over which the air is fed into the vessel. Next, by dividing this air flow rate by the cross-sectional area of a path to introduce air into the vessel such as the air jet flow path 3, the air speed at which the impact is applied to the freeze-dried composition (freeze-dried cake) can be calculated.
Air speed (cm/sec)=air flow rate (ml=cm³/sec)+cross-sectional area of air introduction flow path (cm²)

Specifically, in the case for example of a jet type dry powder inhaler designed such that the bore of the air jet flow path 3 is 1.2 mm, the bore of the discharge flow path is 1.8 mm, and the amount of air stored in the bellows body 10 is about 20 ml, in the case that the amount of air of about 20 ml stored in the bellows body 10 is forcibly introduced onto the freeze-dried composition in the vessel from the air jet flow path 3 in about 0.5 seconds, the air flow rate becomes about 40 ml/sec. Dividing this value by the cross-sectional area of the air introduction flow path (the air jet flow path) (0.06×0.06×3.14=0.0113 cm²), gives 3540 cm/sec. The air speed is thus about 35 m/sec.

Moreover, with the self-inhaling type dry powder inhalers shown later as Embodiments 2, 3 and 4, a mechanism is adopted in which air flowing in from an air introduction flow path 17 applies an impact to the freeze-dried cake, and then the resulting fine particles are discharged from a suction flow path 16; the bores of the air introduction flow path 17 and the suction flow path 16 thus stipulate the flow rate of the air flowing through the paths. The air speed applied to the freeze-dried composition in the vessel can thus be calculated by measuring the flow rate of the air flowing through the air introduction flow path 17 and dividing this by the cross-sectional area of the air introduction flow path 17.
Air speed (cm/sec)=air flow rate (ml=cm³/sec)+cross-sectional area of air introduction flow path 17 (cm²)

Specifically, the flow rate of the air flowing through the air introduction flow path 17 can be measured by installing the dry powder inhaler including the vessel in the slot part of apparatus A (a twin impinger: made by Copley, UK) as mentioned in the European Pharmacopoeia (Third Edition Supplement 2001, p 113-115), and using a flow meter (KOFLOC DPM-3).

For example, with a self-inhaling type dry powder inhaler designed such that the bore of the air introduction flow path 17 is 1.99 mm and the bore of the suction flow path is 1.99 mm, in the case that the air flow rate flowing through the air introduction flow path 17 measured using the flow meter (KOFLOC DPM-3) was 17.7 L/min, i.e. 295 ml/sec, the air speed can be obtained by dividing this value by the cross-sectional area of the air introduction flow path 17 (0.0995×0.0995×3.14=0.0311 cm²) (9486 cm/sec, i.e. 95 m/sec).

Moreover, at least 17 ml/sec can be given as an example of the flow rate of the air applied to the freeze-dried composition filled in the vessel. The air flow rate is preferably at least 20 ml/sec, more preferably at least 25 ml/sec. Here there is no particular limitation on the upper limit of the air flow rate, but an example of 900 L/min can be given. This upper limit is preferably 15 L/sec, more preferably 10 L/sec, yet more preferably 5 L/sec, still more preferably 4 L/sec, in particular preferably 3 L/sec. Specifically, the flow rate should be in a range constituted from a lower limit and an upper limit selected as appropriate from the above, with there being no particular limitation; nevertheless, 17 ml/sec to 15 L/sec, 20 ml/sec to 10 L/sec, 20 ml/sec to 5 L/sec, 20 ml/sec to 4 L/sec, 20 ml/sec to 3 L/sec, and 25 ml/sec to 3 L/sec, can be given as examples of the range.

Moreover, as means for raising the impact pressure of the air introduced from the outside, the dry powder inhaler used in the present invention can have means for discharging air from a discharge port, as explained in detail below, preferably with a small bore, of a flow path close to the freeze-dried composition housed at the bottom of the vessel, for example a needle part having an air introduction flow path or an air jet flow path as described later in the embodiments. Regarding the bore of the discharge port of the flow path, the preferable range varies according to the size of the vessel and so on, with there being no particular limitations; nevertheless, the bore can be in a range of 0.3 to 10 mm, preferably 0.5 to 5 mm, more preferably 0.8 to 5 mm, much more preferably 1 to 4 mm.

The freeze-dried composition housed in a non-powder form in the vessel can be made into fine particles by introducing air into the vessel. Here, the extent of making into fine particles should be such that the particle diameter is suitable for transpulmonary administration; a particle diameter of 10 μm or less, preferably 5 μm or less, can be given as an example. The proportion of effective particles (fine particle fraction) is at least 10%, preferably at least 20%, more preferably 25%, yet more preferably at least 30%, and in particular preferably at least 35%.

According to the system of the invention, by introducing air into the vessel using the dry powder inhaler for applying an air impact having an air speed of at least 1 m/sec and an air flow rate of at least 17 ml/sec to the freeze-dried composition in the vessel, a dry powdered interferon-γ preparation having a particle size suitable for transpulmonary administration can be obtained. Furthermore, the system allows transpulmonary administration of the obtained dry powdered interferon-γ preparation directly to a user by inhalation. Therefore, the dry powder inhalation system for transpulmonary administration of the present invention is a system for producing a dry powdered interferon-γ preparation suitable for transpulmonary administration and, at the same time, a system for transpulmonarily administering the dry powder preparation to a user.

(III) Method of Manufacturing a Dry Powdered Interferon-γ Preparation

Moreover, the present invention relates to a method of manufacturing a dry powdered interferon-γ preparation comprising fine particles with a particle diameter suitable for transpulmonary administration (dry powdered interferon-γ preparation for transpulmonary administration) by inhalation, by making a freeze-dried interferon-γ composition containing a single dose of interferon-γ that has been housed in a vessel into fine particles. The manufacturing method can be implemented by applying a predetermined air impact to the freeze-dried interferon-γ composition housed in the vessel. Specifically, the method of manufacturing the dry powdered interferon-γ preparation of the present invention can be carried out by applying an air impact having an air speed of at least 1 m/sec and an air flow rate of at least 17 ml/sec to the above-mentioned freeze-dried interferon-γ composition for transpulmonary administration of the present invention. Thereby, the freeze-dried composition can be made into a dry powdered interferon-γ preparation having a mean particle diameter of 10 microns or less, and preferably 5 microns or less or a fine particle fraction of 10% or more, preferably 20% or more, more preferably 25% or more, and still more preferably 30% or more. The method of applying the air impact to the freeze-dried interferon-γ composition for transpulmonary administration is not limited; however, the above-mentioned dry powder inhaler used for the above-mentioned dry powder interferon-γ inhalation system for transpulmonary administration of the present invention is preferably used.

It is preferable that the manufacturing method be implemented by introducing air capable of applying the above-described air impact to the freeze-dried composition into the vessel housing to the freeze-dried interferon-γ composition for transpulmonary administration.

The method of manufacturing the dry powdered interferon-γ preparation of the present invention is characterized in that a patient administering the dry powdered preparation can prepare by him/herself the powdered preparation at the time of use (inhalation) by making the freeze-dried interferon-γ composition housed in a vessel into fine particles having a particle diameter suitable for transpulmonary administration.

(IV) Transpulmonary Administration Method

The present invention further relates to a transpulmonary administration method comprising making a freeze-dried interferon-γ composition for transpulmonary administration containing a single dose of interferon-γ into fine particles suitable for transpulmonary administration at the time of usage (administration), and administering the resulting dry powdered interferon-γ preparation in a powder form with fine particles by inhalation. The transpulmonary administration method can be carried out using the above-described dry powder interferon-γ inhalation system for transpulmonary administration of the present invention comprising the vessel housing the above-described freeze-dried interferon-γ composition and the dry powder inhaler used for the above-mentioned dry powder interferon-γ inhalation system for transpulmonary administration.

(V) Use of a Freeze-Dried Interferon-γ Composition for Transpulmonary Administration

The present invention also relates to use of a freeze-dried interferon-γ composition for transpulmonary administration by inhalation.

The present invention relates to use of a freeze-dried interferon-γ composition for manufacture of a dry powdered interferon-γ preparation for transpulmonary administration by inhalation.

### EXAMPLES

Following is a detailed description of the present invention, citing examples; however, the present invention is not limited to these examples.

In the following examples, the disintegration index of the non-powder-form freeze-dried composition (freeze-dried cake) of the present invention, and the fine particle fraction (%), which is an indicator for evaluating the delivery into the lungs of the dry powdered preparation produced, were calculated in accordance with the following methods.

<Calculation of Disintegration Index>

1.0 ml of n-hexane is instilled gently down the wall of the vessel into the prepared non-powder-form freeze-dried composition (freeze-dried cake), and agitation is carried out for about 10 seconds at 3,000 rpm using an Automatic Lab-Mixer NS-8 (made by Pasolina). The mixture obtained is put into a UV cell (made by Shimadzu GLC Center) of optical path length 1 mm and optical path width 10 mm, and then the turbidity of the mixture is measured immediately at a measurement wavelength of 500 nm using a spectrophotometer (UV-240, made by Shimadzu Corporation). The value obtained by dividing the turbidity obtained by the total formulation amount (the total amount (weight) of the active ingredient and the carrier) is taken as the disintegration index.

<Calculation of Fine Particle Fraction>

A vessel filled with the prepared non-powder-form freeze-dried composition is installed into the dry powder inhaler, and using the device a prescribed air impact is applied on the composition, and the fine powdered preparation thus produced is discharged directly into Apparatus A (a twin impinger: made by Copley, UK) as mentioned in the European Pharmacopoeia (Third Edition Supplement 2001, p 113-115). After this, the solvents in stage 1 and stage 2 of the apparatus are respectively collected, and the active ingredient contained in each solvent in the stage 1 or stage 2 is assayed using an appropriate method in accordance with the type of active ingredient in the freeze-dried composition, for example a bioassay method or HPLC (see the report of Lucas et al. (Pharm. Res., 15 (4), 562-569 (1998)) and the report of Iida et al. (Yakugaku Zasshi, 119 (10), 752-762 (1999)). The fraction that can be expected to be delivered into the lungs is that in stage 2 (the aerodynamic diameter of particles recovered in this fraction is 6.4 μm or less); the proportion of the active ingredient that reaches stage 2 and is recovered here is generally called the fine particle fraction (the amount that can be expected to reach the lungs), and is taken as a yardstick for evaluating the suitability as an inhalation for transpulmonary administration.

In Examples and Comparative Examples given below, the active ingredients contained in stage 1 and stage 2 were quantitated, and the weight amount of the active ingredient in stage 2 was divided by the total weight amount of the active ingredients jetted out (the total weight amount of the active ingredients contained in stage 1 and stage 2: hereinafter also referred to as “Stage 1+Stage 2”) to calculate fine particles fraction. Moreover, as a rule in the European Pharmacopoeia, when using the twin impinger (made by Copley, UK), it is stipulated that suction is carried out at an air suction flow rate of 60 L/min, i.e. 1 L/sec, and hence in the examples and comparative examples below this was followed.

### Embodiment 1

### Dry Powder Inhaler (Jet Type 1)

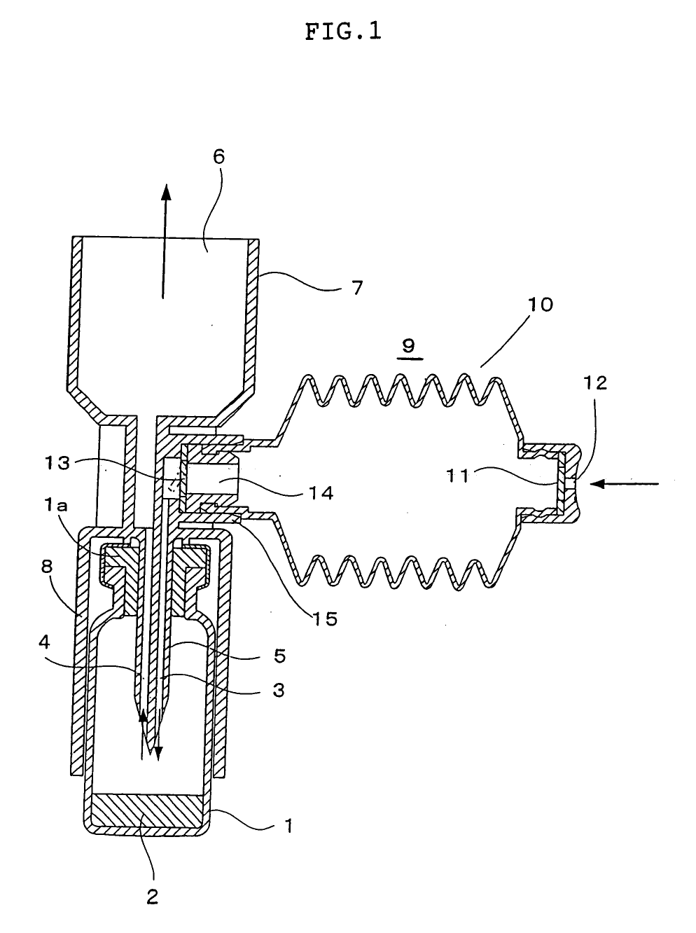

A description of an embodiment of the jet type dry powder inhaler used in the dry powdered interferon-γ inhalation system for transpulmonary administration of the present invention will now be given using FIG. 1.

The dry powder inhaler is an air jet type apparatus for breaking down into fine particles and delivering into the lungs a unit or a plurality of doses of anon-powder-form freeze-dried composition 2 housed at the bottom of a vessel 1, and comprises a needle 5 that has an air jet flow path 3 and a discharge flow path 4, an air intake member 7 that has an inhalation port 6 and is attached to a base end of the needle part 5, a tubular safety cover 8 that surrounds the needle part 5 and also holds the vessel 1, and air pressure-feeding means 9.

The air pressure-feeding means 9 is manually operated and comprises a tubular bellows body 10. An intake port 12 equipped with an intake valve 11, and a discharge port 14 equipped with a discharge valve 13 are provided in the bellows body 10. The discharge port 14 is attached to a connecting port 15 formed at the base end of the air jet flow path 3 of the needle part 5, and communicates with the air jet flow path 3. By applying a compressive force to the bellows body 10 and thus contracting the bellows body 10 in a state in which the intake valve 11 is closed, the discharge valve 13 is opened, and air in the bellows body 10 is discharged into the vessel 1 from the discharge port 14 via the air jet flow path 3. When the compressive force is released, on the other hand, the bellows body 10 expands due to the elastic restoring force of the bellows body 10, and in a state in which the discharge valve 13 is closed, the intake valve 11 opens, and air is introduced into the bellows body 10.

When using the dry powder inhaler, as shown in FIG. 1, the vessel 1 is inserted into the tubular safety cover 8, and a stopper 1a of the vessel 1 is pierced by the needle part 5, thus communicating the air jet flow path 3 and the discharge flow path 4 with the inside of the vessel 1. In this state, if the bellows body 10 of the air pressure-feeding means 9 is contracted to discharge air from the discharge port 14, then this air passes through the air jet flow path 3 and is jetted out from the tip of the needle part 5 towards the freeze-dried composition 2 in the vessel, and due to the resulting air impact the freeze-dried composition 2 becomes fine particles, which then pass through the discharge flow path 4 of the needle part 5 and are discharged from the inhalation port 6 of the air intake member 7. The user (patient) inhales these fine particles from the inhalation port 6 of the air intake member, whereupon the fine particles of the freeze-dried composition 2 are delivered into the lungs of the user (patient). The material of the stopper of the vessel for use in the invention is not limited, and can be selected from materials usually used for a stopper of a vessel for holding a drug or compound, such as rubber, plastic, aluminum or the like.

With this jet type dry powder inhaler, the air jet amount is set to be about 20 ml, the volume of the vessel about 5 ml, the bore (diameter) of the air jet flow path 3 about 1.2 mm, and the bore (diameter) of the discharge flow path 4 about 1.8 mm.

Note, however, that there is no limitation to this. The preferable range for the bores of the air jet flow path 3 and the discharge flow path 4 varies according to the size of the vessel and so on. These bores can be selected as appropriate from a range of 0.3 to 10 mm, preferably 0.3 to 7 mm, more preferably 0.5 to 5 mm.

Moreover, regarding the air pressure-feeding means 9, the discharge amount of fine particles required for administration by inhalation can be adjusted by adjusting the speed of compression of the bellows body 10. Adjustment can also be carried out by such air jet such that most of the freeze-dried composition 2 is broken down into fine particles.

### Embodiment 2

### Dry Powder Inhaler (Self-Inhaling Type 1)

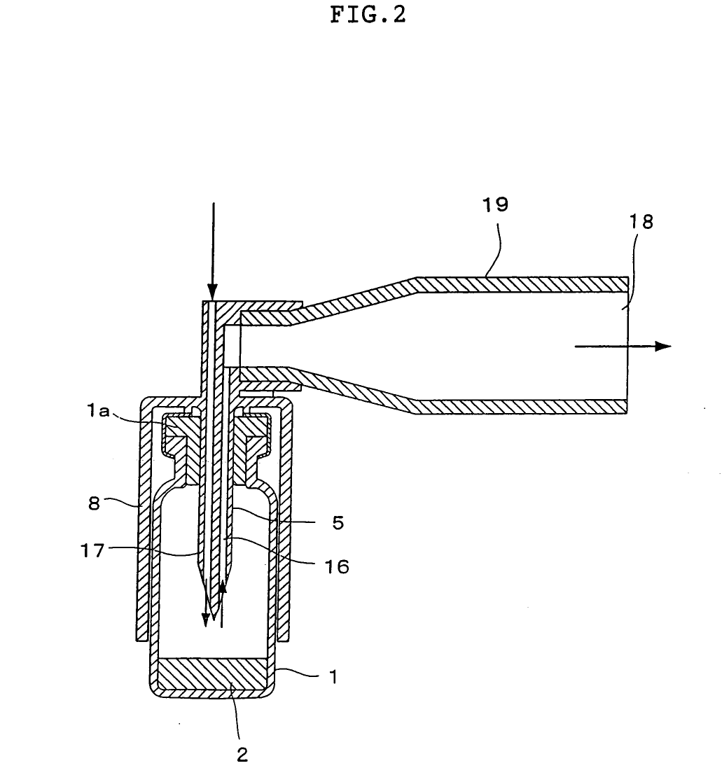

A description of an embodiment (first embodiment) of the self-inhaling type dry powder inhaler used in the dry powdered interferon-γ inhalation system for transpulmonary administration of the present invention will now be given using FIG. 2. The dry powder inhaler shown in FIG. 2 comprises a needle part 5 having a suction flow path 16 and an air introduction flow path 17, a tubular safety cover 8, and an air intake member 19 that has an inhalation port 18 and communicates with the suction flow path 16. The air intake member 19 is connected to the base end of the suction flow path 16 of the needle part 5.

When using the dry powder inhaler, as shown in FIG. 2, the vessel 1 is inserted into the tubular safety cover 8, and an stopper 1a of the vessel 1 is pierced by the needle part 5, thus communicating the suction flow path 16 and the air introduction flow path 17 with the inside of the vessel 1. In this state, through the inhalation pressure of the user (patient), air in the vessel 1 is sucked in from the inhalation port 18 via the suction flow path 16, and at the same time outside air flows into the vessel 1, which is now at a negative pressure, from the air introduction flow path 17. At this time, the freeze-dried composition 2 is made into fine particles through the air impact acting on the freeze-dried composition 2, and the fine particles produced are delivered into the user's (patient's) lungs from the inhalation port 18 via the suction flow path 16.

Moreover, with this dry powder inhaler, setting is carried out such that most of the freeze-dried composition 2 is made into fine particles and discharged from the inhalation port 18 through one inhalation of the user (patient). It is considered that the air flow rate of one inhalation of the user (patient) is 5 to 300 L/min, preferably 10 to 200 L/min, more preferably 10 to 100 L/min, but the design of the self-inhaling type dry powder inhaler of the present invention is modified as appropriate in accordance with the respiratory ability of the user (patient) using the device. With the dry powder inhaler shown in FIG. 2, in accordance with the respiratory ability of the user (patient) in question, the volume of the vessel has been set to about 10 ml, and the bores of the air introduction flow path 17 and the suction flow path 16 to about 1.5 mm. As a result, the settings are such that the freeze-dried composition 2 is made into fine particles and discharged from the inhalation port 18 with virtually none left behind through one inhalation of the user (patient).

### Embodiment 3

### Dry Powder Inhaler (Self-Inhaling Type 2)

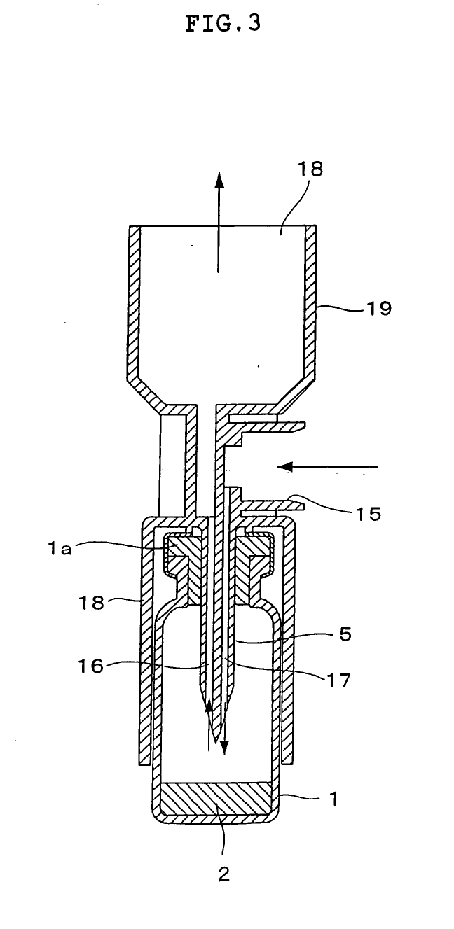

A description of an embodiment (second embodiment) of the self-inhaling type dry powder inhaler used in the dry powdered interferon-γ inhalation system for transpulmonary administration of the present invention will now be given using FIG. 3. The dry powder inhaler shown in FIG. 3 is the same as the jet type dry powder inhaler shown in FIG. 1 with the bellows body 10 used for pressure-feeding air removed from the connecting port 15. The discharge flow path 4 of the jet type dry powder inhaler of FIG. 1 corresponds to a suction flow path 16, the air jet flow path 3 to an air introduction flow path 17, and the air intake member 7 having the inhalation port 6 to an air intake member 19 having an inhalation port 18.

When using the self-inhaling type dry powder inhaler in question, the main points are the same as with the dry powder inhaler shown in FIG. 2. Through the inhalation pressure of the user (patient), air in the vessel 1 is sucked in from the inhalation port 18 via the suction flow path 16, and at the same time outside air flows into the vessel 1, which is now at a negative pressure, from the air introduction flow path 17. The freeze-dried composition 2 is made into fine particles through the air impact produced accompanying this inflow of air. The fine particles produced are then delivered into the user (patient's) lungs from the inhalation port 18. As mentioned before, the air flow rate for one inhalation of the user (patient) is generally in a range of 5 to 300 L/minute; however, with the dry powder inhaler shown in FIG. 3, in accordance with the respiratory ability of the user (patient) in question, the volume of the vessel was set to about 5 ml, the bore (diameter) of the air introduction flow path 17 to about 1.2 mm, and the bore (diameter) of the suction flow path 16 to about 1.8 mm. As a result, the settings are such that most of the freeze-dried composition 2 is made into fine particles and discharged from the inhalation port 18 through one inhalation of the user (patient).

If the self-inhaling type dry powder inhaler is constituted in this way, then by detachably installing air pressure-feeding means 9 such as a bellows body 10 into the connecting port 15, the self-inhaling type dry powder inhaler can be changed into a jet type. A single dry powder inhaler can thus be used as either a self-inhaling type or a jet type as desired.

Each of the above dry powder inhalers of the present invention, regardless of whether it is a self-inhaling type or a jet type, can be constituted such that it is possible to select and set the size of the air impact such that the freeze-dried composition becomes fine particles of mean particle diameter 10 microns or less, preferably 5 microns or less, and flies out with almost none left behind.

### Embodiment 4

### Dry Powder Inhaler (Self-Inhaling Type 3)

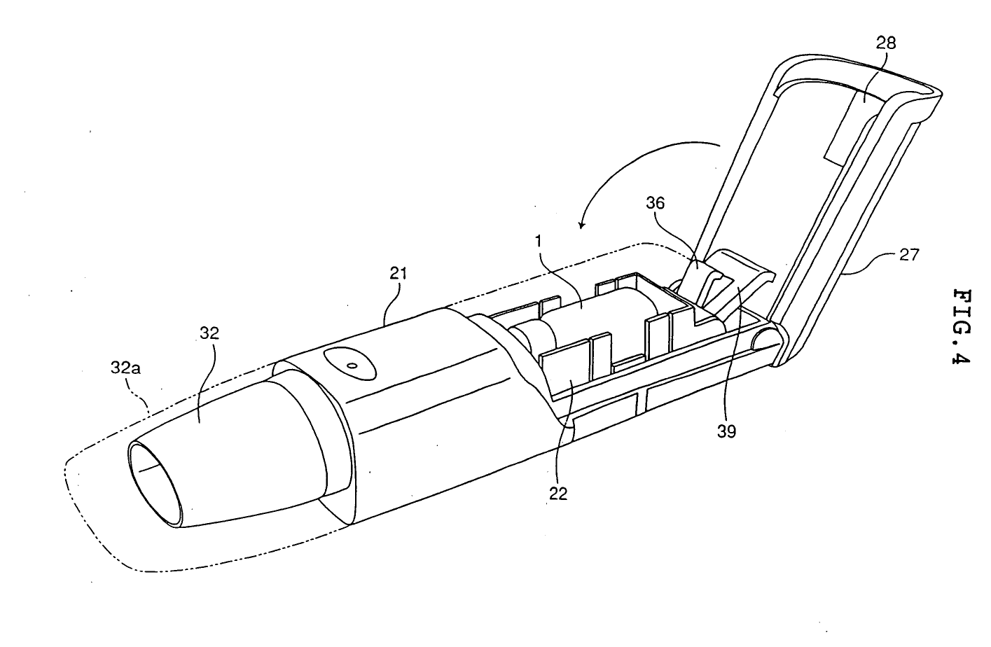

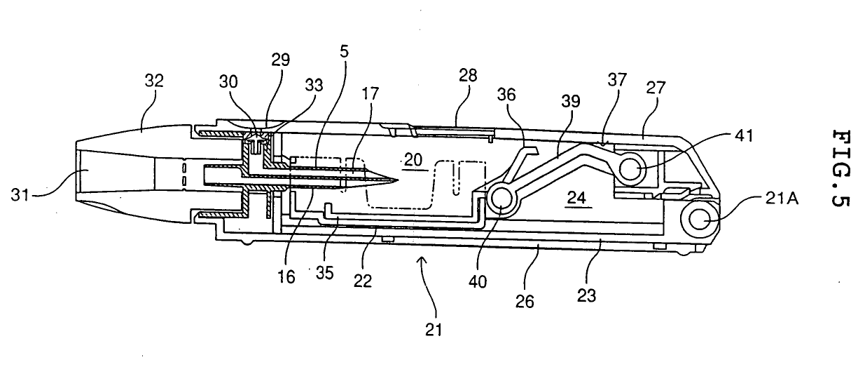

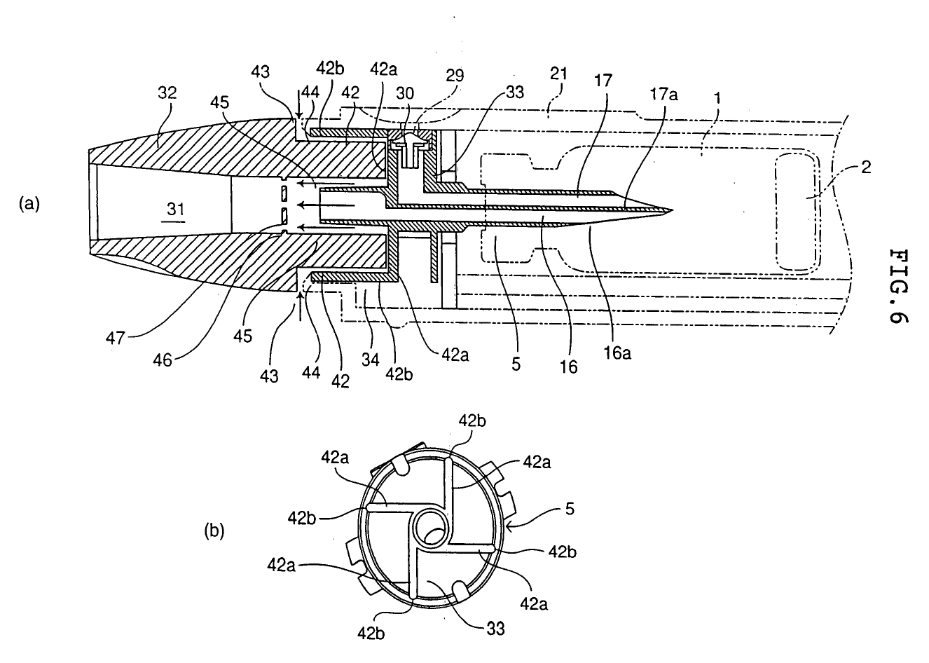

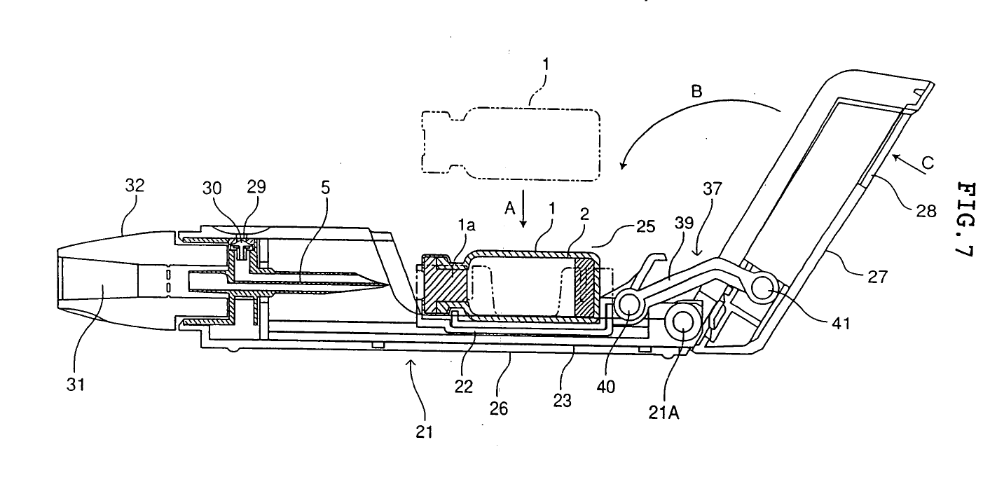

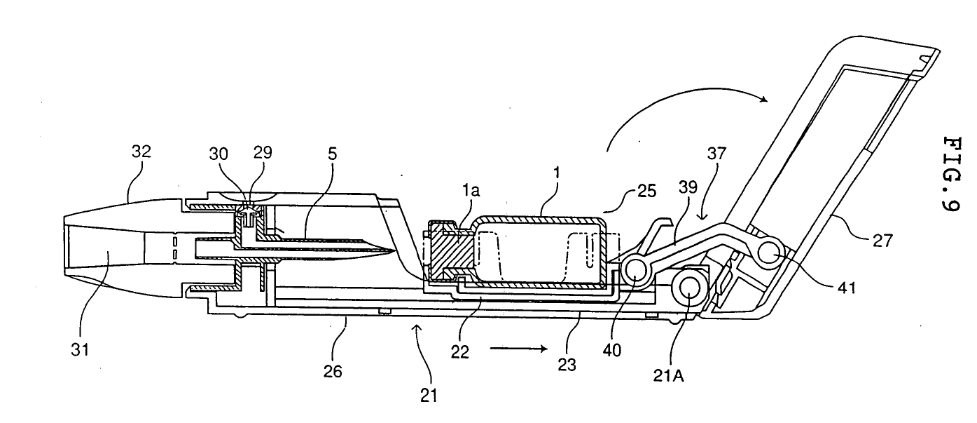

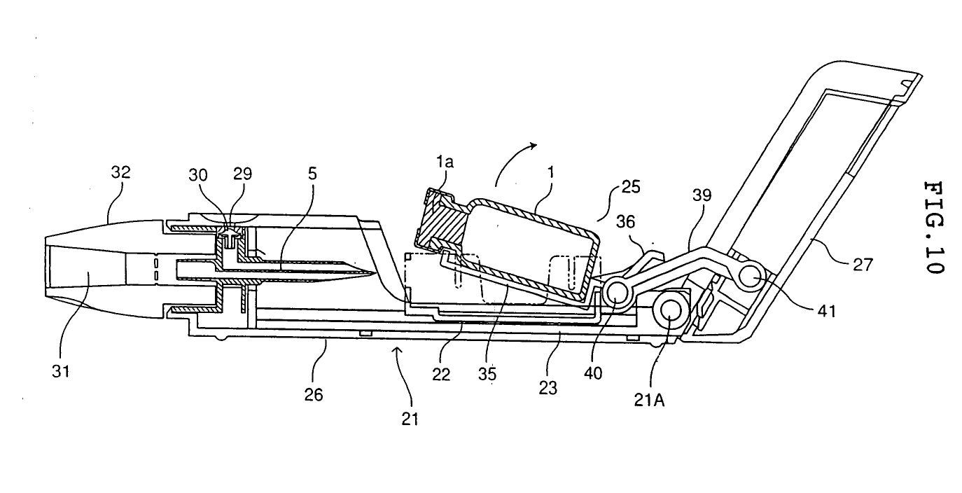

A description of an embodiment (third embodiment) of the self-inhaling type dry powder inhaler used in the dry powdered interferon-γ preparation for transpulmonary administration of the present invention will now be given using FIGS. 4 to 10. FIG. 4 is a perspective view showing the dry powder inhaler, and FIG. 5 is a sectional view showing the dry powder inhaler. Moreover, FIG. 6(a) is a partial sectional view showing a needle part 5 and a suction port 31 of the dry powder inhaler, and (b) is a side view of the needle part 5. Furthermore, FIGS. 7 to 10 are sectional views for explaining the operation of the dry powder inhaler.

The dry powder inhaler comprises a needle part 5 in which are formed a suction flow path 16 and an air introduction flow path 17, a holder part 22 for holding a vessel 1, a housing chamber 20 for housing the vessel 1 via the holder part 22, a guide part 23 provided in the housing chamber 20 for guiding the holder part 22 in the axial direction of the needle part 5, and a holder operating part 24 for advancing and retreating the holder part 22 along the guide part 23; these are all housed in a tubular housing 21. Moreover, a mouthpiece 32 that has a suction port 31 and communicates with the suction flow path 16 of the needle part 5 is provided at a tip of the housing 21.

As shown in FIG. 7, in detail the housing 21 is formed from a housing main body 26 in which is formed a removal/insertion port 25 in a position in which the holder part 22 is retreated, and a lid 27 that opens and closes the removal/insertion port 25. The lid 27 is connected to the housing main body 26 by a hinge 21A, and a window 28 for verifying whether the vessel 1 has been loaded is provided in the lid 27.

An introduction port 29 for introducing outside air is provided in a wall of the housing 21, and a check valve 30 is installed at the introduction port 29. Moreover, the mouthpiece 32 is provided at the tip of the housing 21. The suction port 31 of the mouthpiece 32 is covered by a cap 32a when the dry powder inhaler is not being used.

A flange-shaped partition part 33 is formed at the base end of the needle part 5, and an end of the air introduction flow path 17 passes through the partition part 33 and opens out in an outer peripheral direction of the partition part 33. Moreover, a peripheral wall part 34 extends from an outer rim part of the partition part 33 towards the suction port 31 of the mouthpiece 32. The needle part 5 is installed into the housing 21 by fitting the partition part 33 into the tip part of the housing 21. Through this installation, the axial direction of the housing 21 and the axial direction of the needle part 5 are aligned with one another.

A remover 35 for lifting the vessel 1 up from the base of the holder part 22 and removing the vessel 1 is attached to the holder part 22, and a lever 36 for lifting the vessel 1 up is formed on the remover 35.

The holder operating part 24 comprises a mechanism part 37 for moving the holder part 22 back and forth along the axial direction of the housing 21, and an operating lever for operating the mechanism part 37. The mechanism part 37 comprises a connector 39. One end of the connector 39 is connected to the holder part 22 by a hinge 40, and the other end of the connector 39 is connected to the lid 27 by a hinge 41. The lid 27 is also used as the above-mentioned operating lever. By opening and closing the lid 27, the holder part 22 is advanced and retreated along the guide part 23.

The point where force is applied for pushing down the lid 27 is shown by the arrow C in FIG. 7. That is, the distance from the hinge 21A to the point of action is made to be longer than the distance from the hinge 21A to the hinge 41. As a result, through the lever principle, the lid (operating lever) 27 can be operated by a force smaller than the force necessary to pierce the stopper 1a of the vessel 1 with the needle part 5.

Moreover, as shown in FIG. 6, second introduction paths 42 for supplementary introduction of air are formed in the dry powder inhaler. When sucking the freeze-dried composition that has been made into a powder from the mouthpiece 32, outside air passes through these second introduction paths 42 and flows to the suction port 31 of the mouthpiece 32. As a result, the dry powder inhaler can be used without imposing a burden even by a user (patient) having reduced pulmonary capacity or a child patient. Note that the second introduction paths 42 may be omitted.

Introduction grooves 42a are provided in the partition part 33 of the needle part 5 and introduction grooves 42b are provided in the peripheral wall part 34. By fitting the mouthpiece 32 into the peripheral wall part 34 of the needle part 5, the second introduction paths 42 are thus formed from the mouthpiece 32 and the introduction grooves 42a and 42b.

A slight gap 43 is formed between the mouthpiece 32 and the housing 21, and one end 44 of the second introduction paths 42 opens out to the outside via the gap 43, while the other end 45 of the second introduction paths 42 opens out into the suction port 31 of the mouthpiece 32.

Moreover, as shown in FIG. 6, a wall 47 having vent holes 46 is provided in the suction port 31. Consequently, even in the case that the air impact applied to the freeze-dried composition 2 is small due to a lack of suction force or the like, and part of the freeze-dried composition 2 is not made into a powder, the non-powder part can be made into a powder when passing through the vent holes 46 of the wall 47.

Moreover, as shown in FIG. 6(a), a tip opening 17a of the air introduction flow path 17 of the needle part 5 is made to be closer to the freeze-dried composition 2 than a tip opening 16a of the suction flow path 16. As a result, dropping of the flow speed of the air that flows into the vessel 1 from the tip opening 17a of the air introduction flow path 17 can be suppressed as much as possible, and hence an effective air impact can be applied to the freeze-dried composition 2. Moreover, because the tip opening 16a of the suction flow path 16 of the needle part 5 is further from the freeze-dried composition 2 than the tip opening 17a of the air introduction flow path 17, making of the freeze-dried composition 2 can be made to into a fine powder in the vessel 1 as much as possible before being sucked into the air introduction flow path 16 of the needle part 5.

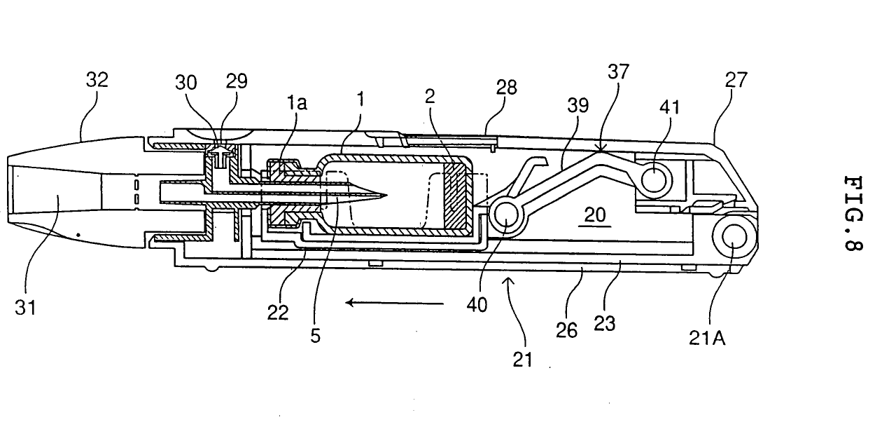

The dry powder inhaler is used as follows. Firstly, the lid 27 is lifted up to open the removal/insertion port 25 of the housing 21 as in FIG. 7, whereby the holder part 22 is pulled backwards to reach the removal/insertion port 25 of the housing 21. Next, the vessel 1 is installed in the holder part 22 with the stopper 1a facing forwards. Next, the lid 27 is pushed down to close the removal/insertion port 25 of the housing 21 as in FIG. 8, whereby the holder part 22 is pushed towards the needle part 5 by the connector 39, and the stopper 1a of the vessel 1 is pierced by the tip of the needle part 5, thus communicating the suction flow path 16 and the air introduction flow path 17 of the needle part 5 with the inside of the vessel 1. Next, air in the vessel 1 is sucked from the suction port 31 of the mouthpiece 32 through the suction flow path 16 of the needle part 5 by the inhalation pressure of the user (patient). At this time, the inside of the vessel 1 becomes a negative pressure and the check valve 30 opens, and outside air flows into the vessel 1 through the air introduction flow path 17 of the needle part 5. As a result, an air impact is generated in the vessel 1 and the freeze-dried composition 2 is broken down into fine particles, and the fine particles prepared are delivered into the user's (patient's) lungs from the suction port 31 via the suction flow path 16. After use, the lid 27 is lifted up to pull the holder part 22 back up to the removal/insertion port 25 of the housing 21, and then the remover 35 is lifted up by the lever 36 and the vessel 1 is removed from the holder part 22.

Even if air is conversely blown into the vessel 1 from the suction port 31 of the mouthpiece 32, discharge to the outside of the freeze-dried composition 2 made into fine particles is prevented by the check valve 30.

As mentioned before, the air flow rate of one inhalation of the user (patient) is generally in a range of 5 to 300 L/min, but with the dry powder inhaler shown in FIGS. 4 to 10, in accordance with the respiratory ability of the user (patient), the volume of the vessel 1 has been set to about 5 ml, the bore (diameter) of the air introduction flow path 17 to about 2.5 mm, and the bore (diameter) of the suction flow path 16 to about 2.5 mm. As a result, the settings are such that most of the freeze-dried composition 2 is made into fine particles and discharged from the suction port 31 through one inhalation of the user (patient).

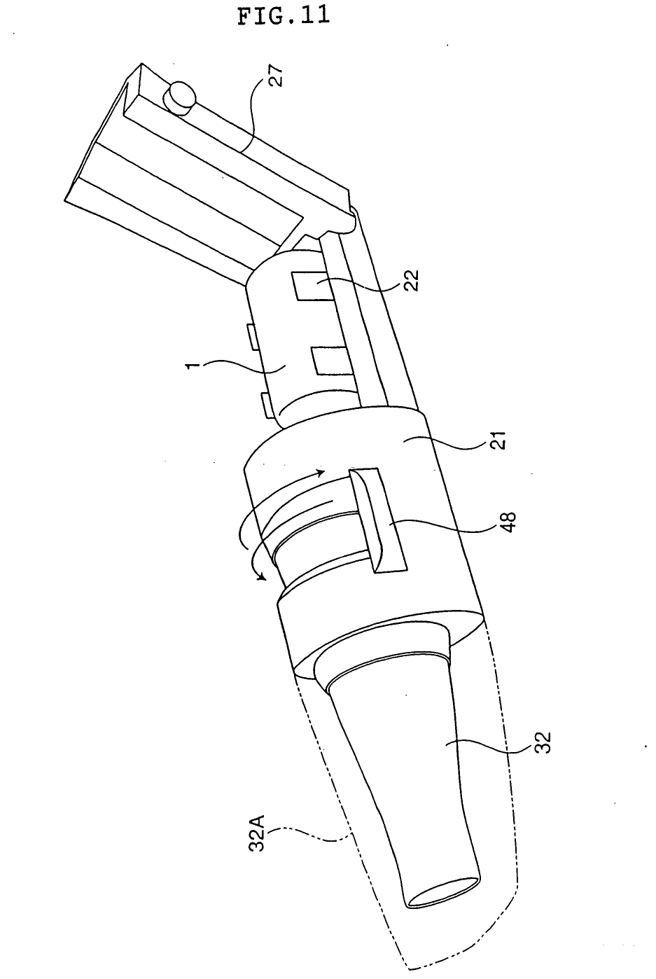

Other embodiments of the dry powder inhaler (self-inhaling type) are shown in FIGS. 11 to 13.

With the dry powder inhaler (self-inhaling type 4) shown in FIG. 11, an operating member 48 is provided so as to be freely rotatable in the circumferential direction of the housing 21 as shown by the arrow. The mechanism part of the holder operating part, which is not shown in the drawing, comprises a spiral groove and a follower that engages into the same; when the operating member 48 is rotated, this rotation is converted to linear movement of the holder part 22 in the axial direction of the needle part 5. Note that the angle of rotation of the operator 48 is about 180°.

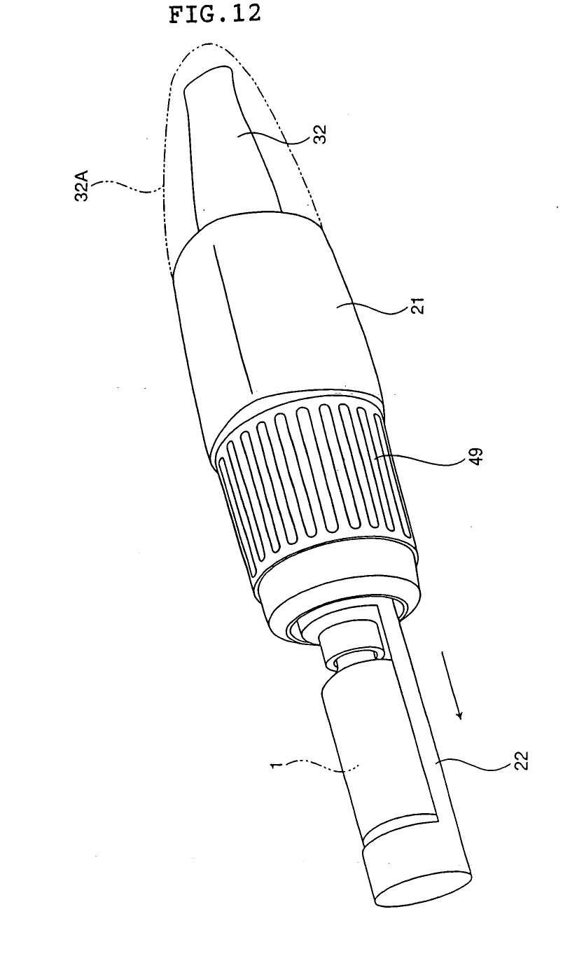

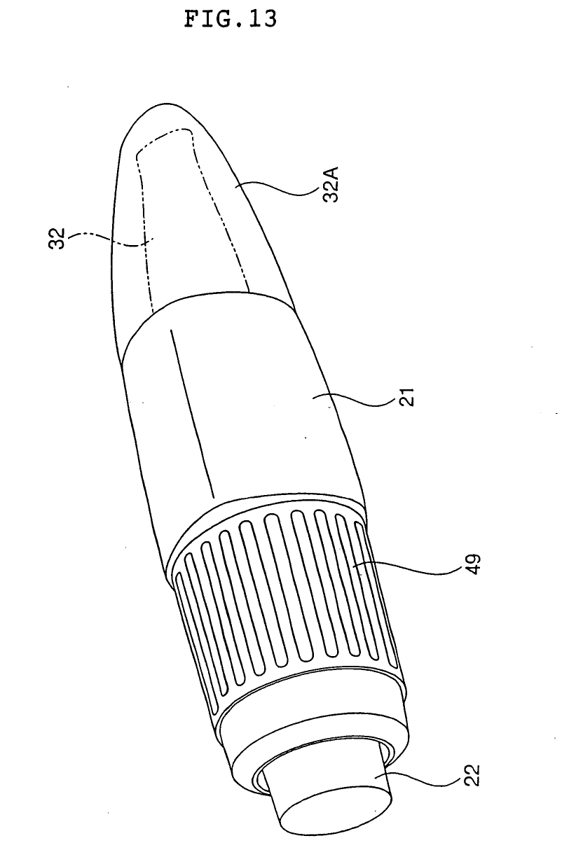

With the dry powder inhaler (self-inhaling type 5) shown in FIG. 12 and FIG. 13, an annular operating member 49 is installed so as to be freely rotatable in the housing 21. The mechanism part of the holder operating part, which is not shown in the drawing, comprises a feed screw; when the operating member 49 is rotated, this rotation is converted to linear movement of the holder part 22 in the axial direction of the needle part 5. The holder part 22 can be withdrawn from the back of the housing 21.

### Examples 1 To 4

An interferon-γ (IFN-γ) stock liquid (potency: 1×10⁷ IU/ml) was desalinated using an ultrafilter membrane (Ultrafree 15, manufactured by Millipore). 100,000 IU of the desalinated IFN-γ obtained and various carriers having the amount shown in Table 1 below were dissolved into distilled water for an injection such that the volume was 0.5 ml and the resultant was filled into vessels (trunk diameter 18 mm), and freeze-drying was carried out using a shelf-type freeze-dryer (Lyovac GT-4, made by Leybold)

### Examples 1 to 4 and Comparative Examples 1 and 2

The disintegration index of the non-powder-form (cake-like) freeze-dried composition (freeze-dried cake) obtained was calculated.

Moreover, to calculate the fine particle fraction (%) of the fine particles for each freeze-dried composition and thus evaluate the efficiency of delivery into the lungs, an air impact arising through an air speed of about 35 m/sec and an air flow rate of about 40 ml/sec was applied to the freeze-dried cake filled into a vessel using the dry powder inhaler, and the resulting powdered fine-particle-form freeze-dried composition was discharged directly into a twin impinger (made by Copley, UK). After this, the solvents in stage 1 and stage 2 were collected, and the IFN-γ in the stage 1 and stage 2 solvents were assayed using a bioassay method. The value obtained by dividing the amount (weight) of IFN-γ obtained in stage 2 by the total amount (weight) of IFN-γ jetted out (stage 1+stage 2) was then calculated as the fine particle fraction (%).

To evaluate stability of IFN-γ of the freeze-dried composition obtained, residual activity of IFN-γ immediately after freeze-drying (hereinafter, referred to as residual activity after freeze-drying) compared to activity (100%) of IFN-γ immediately before freeze-drying, and residual activity of IFN-γ after preserved at 70° C. for two weeks (hereinafter, referred to as residual activity after high-temperature preservation) compared to activity (100%) of IFN-γ immediately after freeze-drying was assayed by bioassay method.

The disintegration index, fine particle fraction (%), residual activity after freeze-drying (%) and residual activity (%) after high-temperature preservation for each freeze-dried composition (Examples 1 to 4 and Comparative Examples 1 and 2) are shown in Table 1.

TABLE 1

| | Ex. 1 | Ex. 2 | Ex. 3 | Ex. 4 | Com. Ex. 1 | Com. Ex. 2 |
|---|---|---|---|---|---|---|
| IFN-γ | 100,000 IU | 100,000 IU | 100,000 IU | 100,000 IU | 100,000 IU | 100,000 IU |
| Phenylalanine | 1 mg | 1 mg | 1 mg | 1 mg | 1 mg | — |
| Arginine | 0.2 mg | 0.5 mg | 1.2 mg | 1.5 mg | — | — |
| hydrochloride | | | | | | |
| Pullulan | — | — | — | — | — | 2 mg |
| Disintegration | 0.269 | 0.251 | 0.235 | 0.247 | 0.232 | 0.001 |
| Index | | | | | | |
| Fine particle | 59% | 55% | 48% | 50% | 77% | 0% |
| fraction | | | | | | |
| Residual | 70% | 77% | 100% | 98% | 56% | —^{#1} |
| activity | | | | | | |
| after | | | | | | |
| freeze-drying | | | | | | |
| Residual | 100% | 100% | 100% | 97% | 21% | —^{#1} |
| activity after | | | | | | |
| high-temperature | | | | | | |
| preservation | | | | | | |

Note:
^{#1}The residual activity after freeze-drying and residual activity after high-temperature preservation for the comparative example 2 were not measured.

The freeze-dried compositions obtained in Examples 1 to 4 and the Comparative Example 1 were in the form of a non-powder cake-like lump (freeze-dried cake) after freeze-drying. As shown in Table 1, the freeze-dried compositions obtained in Examples 1 to 4 and Comparative Example 1 were easily made into fine particles in the vessel by an air impact arising through an air speed of about 35 m/sec and an air flow rate of about 40 ml/sec, and thus obtained a suitable fine particle fraction. Therefore, it was verified that the freeze-dried compositions obtained in Examples 1 to 4 and Comparative Example 1 were possible to produce a powdered preparation suitable for transpulmonary administration. The freeze-dried composition containing pullulan as a carrier was not disintegrated by the air impact, and did not form fine particles.

Moreover, it was verified that the freeze-dried compositions obtained in Examples 1 to 4 maintained a high IFN-γ activity due to the freeze-drying process as compared to the freeze-dried composition containing no hydrophilic amino acids in Comparative Example 1. It was also verified that IFN-γ in the freeze-dried composition containing no hydrophilic amino acids in Comparative Example 1 deactivated under an extremely severe temperature conditions (70° C.) while the freeze-dried compositions containing hydrophobic amino acids and hydrophilic amino acids obtained in Examples 1 to 4 maintained high IFN-γ activity under such temperature conditions.

### Examples 5 to 11

An interferon-γ (IFN-γ) stock liquid (potency: 1×10⁷ IU/ml) was desalinated using an ultrafilter membrane (Ultrafree 15, manufactured by Millipore). 100,000 IU or 1,000,000 IU of the desalinated IFN-γ obtained and various carriers having the amount shown in Table 2 were dissolved into distilled water for an injection such that the total amount was 0.5 ml and the resultant was filled into vessels (trunk diameter 18 mm), and freeze-drying was carried out using a shelf-type freeze-dryer (Lyovac GT-4, made by Leybold) (Examples 5 to 11).

The disintegration index of the non-powder-form freeze-dried composition (freeze-dried cake) obtained was calculated.

Next, a vessel filled with the non-powder-form freeze-dried composition (freeze-dried cake) obtained in Examples 5 to 11 was installed in a jet type dry powder inhaler (having a bellows body 10 capable of supplying an amount of air of about 20 ml; FIG. 1) designed such that the bore of the air jet flow path 3 was 1.2 mm and the bore of the discharge flow path 4 was 1.8 mm. This inhaler was attached to an Aerosizer (made by Amherst Process Instrument, Inc., USA) fitted with an Aerobreather, which is an artificial lung model, and an amount of air of about 20 ml was introduced into the vessel from the inhaler, thus applying an air impact arising through an air speed of about 35 m/sec and an air flow rate of about 40 ml/sec to the freeze-dried cake. As a result, air was introduced from the air jet flow path 3 of the jet type dry powder inhaler into the vessel 1, and it was observed that the non-powder-form freeze-dried composition in the vessel was made into fine particles by the air impact. The particle size distribution of the fine particles was measured using the Aerosizer fitted with the Aerobreather (measurement conditions: breath rate: 60 L/min, breath volume: 1 L, acceleration: 19). The mass median aerodynamic diameter (μm±SD) was then calculated from the particle size distribution of the fine particles jetted out from the inhaler.

The fine particle fraction (%), residual activity after freeze-drying (t) and residual activity (%) after high-temperature preservation for each freeze-dried composition were evaluated in the same manner as in Examples 1 to 4.

The freeze-dried compositions obtained in Examples 5 to 11 were in the form of a non-powder cake-like lump (freeze-dried cake) after freeze-drying. As shown in Table 2, the freeze-dried compositions obtained in Examples 5 to 11, which showed a disintegration index of at least 0.15, were easily made into fine particles in the vessel by an air impact arising through an air speed of about 35 m/sec and an air flow rate of about 40 ml/sec, and thus obtained a fine particle fraction having a mass median aerodynamic diameter of 5 microns or less, and hence it was possible to produce preparations suitable for transpulmonary administration. Each freeze-dried composition showed a favorable fine particle fraction. Moreover, it was verified that the freeze-dried composition obtained in Examples 5 to 11 showed high residual activity after freeze-drying and residual activity after high-temperature preservation, and also maintained high IFN-γ activity even in the preparation of a composition and under conditions of high-temperature preservation.

TABLE 2

| | | | | | | Com. | Com. |
|---|---|---|---|---|---|---|---|
| | Ex. 5 | Ex. 6 | Ex. 7 | Ex. 8 | Ex. 9 | Ex. 10 | Ex. 11 |
| IFN-γ (IU) | 100,000 | 100,000 | 100,000 | 1,000,000 | 1,000,000 | 1,000,000 | 1,000,000 |
| Phenylalanine | 1.2 mg | 1.2 mg | 1.2 mg | 1 mg | 1 mg | 1 mg | — |
| Leucine | 0.3 mg | — | — | 0.3 mg | — | — | — |
| Valine | — | 0.3 mg | — | — | 0.3 mg | — | 0.8 mg |
| Isoleucine | — | — | 0.3 mg | — | — | 0.3 mg | — |
| Arginine | 0.2 mg | 0.2 mg | 0.2 mg | 0.2 mg | 0.2 mg | 0.2 mg | 0.2 mg |
| hydrochloride | | | | | | | |
| Disintegration | 0.191 | 0.190 | 0.181 | 0.316 | 0.293 | 0.281 | 0.150 |
| Index | | | | | | | |
| Mass median | 1.537 ± 1.438 | 1.698 ± 0.542 | 1.874 ± 1.842 | 1.278 ± 0.386 | 1.387 ± 1.591 | 1.964 ± 1.673 | 1.597 ± 1.625 |
| aerodynamic | | | | | | | |
| diameter | | | | | | | |
| (μm ± SD, MMDA) | | | | | | | |
| Fine particle | 67% | 64% | 67% | 85% | 82% | 78% | 70% |
| fraction | | | | | | | |
| Residual activity | 83% | 80% | 84% | 100% | 92% | 97% | 80% |
| after freeze-drying | | | | | | | |
| Residual activity | 93% | 95% | 98% | 93% | 98% | 78% | 87% |
| after high- | | | | | | | |
| temperature | | | | | | | |
| preservation | | | | | | | |

### Examples 12 to 14

An interferon-γ (IFN-γ) stock liquid (potency: 1×10⁷ IU/ml) was desalinated using an ultrafilter membrane (Ultrafree 15, manufactured by Millipore). 100,000 IU of the desalinated IFN-γ thus obtained and various carriers having the amount shown in Table 3 below were dissolved into distilled water for an injection such that the total amount was 0.5 ml. The resultant was filled into vessels (trunk diameter 18 mm), and freeze-drying was carried out using a shelf-type freeze-dryer (Lyovac GT-4, made by Leybold) (Examples 12 to 14).

The disintegration index of the non-powder-form freeze-dried composition (freeze-dried cake) obtained was calculated.

The mass median aerodynamic diameter (μm±SD) was calculated for each freeze-dried composition in the same manner as in Examples 5 to 11. Residual activity after freeze-drying (%) and residual activity (%) after high-temperature preservation for each freeze-dried composition were evaluated in the same manner as in Examples 1 to 4.

The freeze-dried compositions obtained in Examples 12 to 14 were in the form of a non-powder cake-like lump (freeze-dried cake) after freeze-drying. As shown in Table 3, the freeze-dried compositions obtained in Examples 12 to 14, which showed a disintegration index of at least 0.25, were easily made into fine particles in the vessel by an air impact arising through an air speed of about 35 m/sec and an air flow rate of about 40 ml/sec, and thus obtained fine particle fraction having a mass median aerodynamic diameter of 5 microns or less, and hence it was possible to produce a preparation suitable for transpulmonary administration. Moreover, it was verified that the freeze-dried compositions obtained in Examples 12 to 14 showed high residual activity after freeze-drying and residual activity after high-temperature preservation, and also maintained high IFN-γ activity even in the preparation of a composition and under conditions of high-temperature preservation.

TABLE 3

| | Ex. 12 | | Ex. 13 | | Ex. 14 | |
|---|---|---|---|---|---|---|
| IFN-γ | 1,000,000 | IU | 1,000,000 | IU | 1,000,000 | IU |
| Phenylalanine | 0.8 | mg | 1 | mg | 1 | mg |

| | | | | | |
|---|---|---|---|---|---|
| Leucine | — | 0.3 | mg | 0.3 | mg |

| | | | | |
|---|---|---|---|---|
| Leucine- | 0.2 | mg | — | — |
| phenylalanine | | | | |

| | | | | |
|---|---|---|---|---|
| Lysine | — | 0.2 | mg | — |

| | | | | |
|---|---|---|---|---|
| Threonine | — | — | 0.2 | mg |

| | | | | |
|---|---|---|---|---|
| Arginine | 0.2 | mg | — | — |
| hydrochloride | | | | |

| | | | |
|---|---|---|---|
| Disintegration index | 0.251 | 0.285 | 0.327 |
| Mass median | 1.578 ± 1.285 | 1.389 ± 1.427 | 1.256 ± 1.223 |
| aerodynamic | | | |
| diameter | | | |
| (μm ± SD, MMDA) | | | |
| Residual | 90% | 83% | 92% |
| activity | | | |
| after | | | |
| freeze-drying | | | |
| Residual | 92% | 85% | 89% |
| activity after | | | |
| high-temperature | | | |
| preservation | | | |

### Example 15

An interferon-γ (IFN-γ) stock liquid (potency: 1×10⁷ IU/ml) was desalinated using an ultrafilter membrane (Ultrafree 15, manufactured by Millipore). 100,000 IU of the desalinated IFN-γ thus obtained and various carriers having the amount shown in Table 4 below were dissolved into mixed solution of distilled water for an injection and ethanol (ethanol concentration: 1 wt %) such that the total amount was 0.5 ml. The resultant was filled into vessels (trunk diameter 18 mm), and freeze-drying was carried out using a shelf-type freeze-dryer (Lyovac GT-4, made by Leybold) (Example 15).

The disintegration index of the non-powder-form freeze-dried composition (freeze-dried cake) obtained in Example 15 was calculated.

Next, a vessel filled with the non-powder-form freeze-dried composition (freeze-dried cake) obtained in Example 15 was installed in a jet type dry powder inhaler (having a bellows body 10 capable of supplying an amount of air of about 50 ml; FIG. 1) designed such that the bore of the air jet flow path 3 was 1.2 mm and the bore of the discharge flow path 4 was 1.8 mm. This inhaler was attached to an Aerosizer (made by Amherst Process Instrument, Inc., USA) fitted with an Aerobreather, which is an artificial lung model, and an amount of air of about 50 ml was introduced into the vessel from the inhaler, thus applying an air impact arising through an air speed of about 89 m/sec and an air flow rate of about 100 ml/sec to the freeze-dried cake. As a result, air was introduced from the air jet flow path of the jet type dry powder inhaler into the vessel, and it was observed that the non-powder-form freeze-dried composition in the vessel was made into fine particles by the air impact. The particle size distribution of the fine particles was measured using the Aerosizer fitted with the Aerobreather (measurement conditions: breath rate: 60 L/min, breath volume: 1 L, acceleration: 19). The mass median aerodynamic diameter (μm±SD) was then calculated from the particle size distribution of the fine particles jetted out from the inhaler.

The residual activity after freeze-drying (%) and residual activity (%) after high-temperature preservation for each freeze-dried composition were evaluated in the same manner as in Examples 1 to 4.

The freeze-dried composition obtained in Example 15 was in the form of a non-powder cake-like lump (freeze-dried cake) after freeze-drying. As shown in Table 4, the freeze-dried composition obtained in Example 15, which showed a disintegration index at least 0.05, was easily made into fine particles in the vessel by an air impact arising through an air speed of about 89 m/sec and an air flow rate of about 100 ml/sec, and thus obtained fine particle fraction having a mass median aerodynamic diameter of 5 microns or less, and hence it was possible to produce a preparation suitable for transpulmonary administration. Moreover, it was verified that the freeze-dried composition obtained in Example 15 showed high residual activity after freeze-drying and residual activity after high-temperature preservation, and also maintained high IFN-γ activity even in the preparation of a composition and under conditions of high-temperature preservation.

TABLE 4

| | Ex. 15 | |
|---|---|---|
| IFN-γ | 1,000,000 | IU |
| Leucyl-valine | 1.3 | mg |
| Arginine | 0.2 | mg |
| hydrochloride | | |

| | |
|---|---|
| Disintegration | 0.053 |
| index | |
| Mass median | 1.983 ± 1.676 |
| aerodynamic | |
| diameter | |
| (μm ± SD, MMDA) | |
| Residual activity | 89% |
| after freeze-drying | |
| Residual activity | 82% |
| after | |
| high-temperature | |
| preservation | |

### Example 16

An interferon-γ (IFN-γ) stock liquid (potency: 1×10⁷ IU/ml) was desalinated using an ultrafilter membrane (Ultrafree 15, manufactured by Millipore). 100,000 IU of the desalinated IFN-γ thus obtained and various carriers having the amount shown in Table 5 were dissolved into distilled water for an injection such that the total amount was 0.5 ml and the resultant was filled into vessels (trunk diameter 18 mm), and freeze-drying was carried out using a shelf-type freeze-dryer (Lyovac GT-4, made by Leybold) (Example 16).

The disintegration index of the non-powder-form freeze-dried composition (freeze-dried cake) obtained in Example 16 was calculated.

Next, a vessel filled with the non-powder-form freeze-dried composition (freeze-dried cake) obtained in Examples 16 was installed in a self-inhaling type dry powder inhaler designed such that the bore of the air jet flow path was 4.0 mm and the bore of the discharge flow path was 4.0 mm. This inhaler was attached to an Aerosizer (made by Amherst Process Instrument, Inc., USA) fitted with an Aerobreather, which is an artificial lung model, and thus applying an air impact arising through an air speed of about 1 m/sec and an air flow rate of about 17 ml/sec to the freeze-dried cake. As a result, air was introduced from the air jet flow path of the jet type dry powder inhaler into the vessel, and it was observed that the non-powder-form freeze-dried composition in the vessel was made into fine particles by the air impact. The particle size distribution of the fine particles was measured using the Aerosizer fitted with the Aerobreather (measurement conditions: breath rate: 60 L/min, breath volume: 1 L, acceleration: 19). The mass median aerodynamic diameter (μm±SD) was then calculated from the particle size distribution of the fine particles jetted out from the inhaler.

The residual activity after freeze-drying (%) and residual activity (%) after high-temperature preservation for each freeze-dried composition were evaluated in the same manner as in Examples 1 to 4.

The freeze-dried composition obtained in Example 16 was in the form of a non-powder cake-like lump (freeze-dried cake) after freeze-drying. As shown in Table 5, the freeze-dried composition obtained in Example 16, which showed a disintegration index at least 0.2, was easily made into fine particles in the vessel by an air impact arising through an air speed of about 1 m/sec and an air flow rate of about 17 ml/sec, and thus obtained fine particle fraction having a mass median aerodynamic diameter of 5 microns or less, and hence it was possible to produce a fine particle powder-form preparation suitable for transpulmonary administration. Moreover, it was verified that the freeze-dried composition obtained in Example 16 showed high residual activity after freeze-drying and residual activity after high-temperature preservation, and also maintained high IFN-γ activity even in the preparation of a composition and under conditions of high-temperature preservation.

TABLE 5

| | Ex. 16 | |
|---|---|---|
| IFN-γ | 1,000,000 | IU |
| Valine | 0.5 | mg |
| Arginine | 0.2 | mg |
| hydrochloride | | |

| | |
|---|---|
| Disintegration | 0.205 |
| index | |
| Mass median | 1.610 ± 1.548 |
| aerodynamic | |
| diameter | |
| (μm ± SD, MMDA) | |
| Residual activity | 82% |
| after freeze-drying | |
| Residual activity | 83% |
| after | |
| high-temperature | |
| preservation | |

### Reference Example 1

The following tests were conducted to determine the effects of salts contained in the freeze-dried composition on powderizeation by air impact.

Interferon-α (IFN-α), various amino acids and citrates (citric acid and sodium citrate) having the amounts shown in Table 6 were dissolved into distilled water for an injection such that the total amount was 0.5 ml. The resultant was filled into vessels (trunk diameter 18 mm), and freeze-drying was carried out using a shelf-type freeze-dryer (Lyovac GT-4, made by Leybold) (Examples 1 to 5). The disintegration index and fine particle fraction of the non-powder-form freeze-dried compositions (freeze-dried cake) obtained were calculated in the same manner as in Examples 1 to 4.

The obtained results were shown in Table 6. As can be seen from Table 6, it was verified that the disintegration index was increased when the content of citrate in the freeze-dried composition was small. Moreover, it was verified that the proportion of effective particles was excellent when the content of citrate in the freeze-dried composition was small.

TABLE 6

| | Ref. Ex. 1 | Ref. Ex. 2 | Ref. Ex. 3 | Ref. Ex. 4 | Ref. Ex. 5 |
|---|---|---|---|---|---|
| IFN-α | 10,000,000 IU | 10,000,000 IU | 10,000,000 IU | 10,000,000 IU | 10,000,000 IU |
| Leucine | 1.8 mg | 1.8 mg | 1.8 mg | 1.8 mg | 1.8 mg |
| Valine | 1.2 mg | 1.2 mg | 1.2 mg | 1.2 mg | 1.2 mg |
| Citrate | — | 0.06 mg | 0.12 mg | 0.24 mg | 0.49 mg |
| Disintegration | 0.237 | 0.245 | 0.218 | 0.207 | 0.198 |
| Index | | | | | |
| Fine particle | 74% | 66% | 65% | 63% | 53% |
| fraction | | | | | |

### Reference Example 2

The following tests were conducted to determine the effects of salts contained in the freeze-dried composition on powderization by air impact.

Interferon-α (IFN-α), various amino acids and phosphates (sodium dihydrogenphosphate dihydrate and disodium hydrogenphosphate dodecahydrate) having the amounts shown in Table 7 were dissolved into distilled water for an injection such that the total amount was 0.5 ml. The resultant was filled into vessels (trunk diameter 18 mm), and freeze-drying was carried out using a shelf-type freeze-dryer (Lyovac GT-4, made by Leybold) (Examples 6 to 8). The disintegration index and fine particle fraction of the non-powder-form freeze-dried compositions (freeze-dried cake) obtained were calculated in the same manner as in Examples 1 to 4.

The obtained results were shown in Table 7. As can be seen from Table 7, it was verified that the disintegration index was increased and the proportion of effective particles became higher when the content of phosphate in the freeze-dried composition was low.

TABLE 7

| | Ref. Ex. 6 | | Ref. Ex. 7 | | Ref. Ex. 8 | |
|---|---|---|---|---|---|---|
| IFN-α | 1,000,000 | IU | 1,000,000 | IU | 1,000,000 | IU |
| Leucine | 1.5 | mg | 1.5 | mg | 1.5 | mg |
| Valine | 1 | mg | 1 | mg | 1 | mg |

| | | | | | |
|---|---|---|---|---|---|
| Phosphate | — | 0.05 | mg | 0.5 | mg |

| | | | |
|---|---|---|---|
| Disintegration | 0.185 | 0.196 | 0.168 |
| Index | | | |
| Fine particle | 59% | 55% | 44% |
| fraction | | | |

The results obtained from Reference Examples 1 and 2 show that salts contained in the non-powder-form freeze-dried composition inhibit the composition from being made into fine particles. Thus, it was verified that the disintegration index was increased and the proportion of effective particles became higher when the content of salts contained in the non-powder-form freeze-dried composition was low. More specifically, the non-powder-form freeze-dried composition which can be prepared by an air impact into fine particles having an excellent proportion of effective particles can be obtained by reducing the concentration of the salts contained in the solution used for freeze-drying.

### INDUSTRIAL APPLICABILITY

The freeze-dried composition for transpulmonary administration of the present invention can be made into fine particles down to the size necessary for delivery into the lungs by an air impact having an air speed of at least 1 m/sec and an air flow rate of at least 17 ml/sec. Therefore, a user (patient) himself/herself can prepare the freeze-dried composition into a powdered preparation comprising fine particles suitable for transpulmonary administration at the time of use (in particular, the time of inhalation) using a simple means.

The proportion of effective particles (fine particle fraction) attained by the freeze-dried composition for transpulmonary administration of the present invention is at least 10%, and can be increased to at least 20%, at least 25%, at least 30% or at least 35%. U.S. Pat. No. 6,153,224 indicates that, with many of prior art dry powder inhalers, the proportion of the active ingredient (particles) to adhere to the lower portions of the lungs is only about 10% of the amount of the active ingredient inhaled. Further, Japanese Unexamined Patent Publication No. 2001-151673 states that the amount of an inhalation powder preparation reaching the lungs (lung reaching proportion) is generally about 10% of the drug discharged from the preparation. Therefore, the freeze-dried interferon-γ composition for transpulmonary administration of the present invention is valuable in that it is capable of achieving a higher proportion of effective particles (fine particle fraction) than prior art powder inhalation preparations.

Prior compositions for transpulmonary administration were hard to handle at the time of preparation because of the fine particle powder form. In contrast, the freeze-dried composition for transpulmonary administration of the present invention is easy to handle because of the cake-like form. In addition, a single dose amount of the composition can be prepared directly in the vessel, which eliminates the need for subdividing the composition into vessels. Therefore, the freeze-dried composition for transpulmonary administration of the present invention can be prepared at high preparatory yield as compared to fine particle powder-form compositions for transpulmonary administration, and moreover, can avoid contamination with impurities due to subdividing the fine particle powder form into vessels.

Moreover, the freeze-dried interferon-γ composition for transpulmonary administration of the present invention can maintain IFN-γ stably. Therefore, the activity of IFN-γ can be maintained at high ratios even when subjected to a freeze-drying process during preparation or a long-period of preservation.

Interferon-γ can be easily administered to the lungs by inhalation with easy according to the dry powder interferon-γ inhalation system for transpulmonary administration of the present invention.

1. A freeze-dried interferon-γ composition for transpulmonary administration having the following properties (i) to (iv):
(i) containing at least one hydrophobic stabilizer selected from the group consisting of hydrophobic amino acids, dipeptides of hydrophobic amino acids, tripeptides of hydrophobic amino acids and derivatives of hydrophobic amino acids and salts thereof; at least one hydrophilic stabilizer selected from the group consisting of hydrophilic amino acids, dipeptides of hydrophilic amino acids, tripeptides of hydrophilic amino acids, derivatives of hydrophilic amino acids and salts thereof; and interferon-γ
(ii) a non-powder cake-like form;
(iii) a disintegration index of 0.015 or more; and
(iv) becoming fine particles having a mean particle diameter of 10 microns or less or a fine particle fraction of 10% or more upon receipt of an air impact having an air speed of at least 1 m/sec and an air flow rate of at least 17 ml/sec.

2. The freeze-dried interferon-γ composition according to claim 1, wherein the hydrophilic stabilizer is at least one selected from the group consisting of basic amino acids, neutral hydroxy amino acids, dipeptides of these amino acids, tripeptides of these amino acids, derivatives of these amino acids and salts thereof.

3. The freeze-dried interferon-γ composition according to claim 1, wherein the hydrophilic stabilizer is at least one selected from the group consisting of basic amino acids, dipeptides of basic amino acids, tripeptides of basic amino acids, derivatives of basic amino acids and salts thereof.

4. The freeze-dried interferon-γ composition according to claim 1, wherein the hydrophilic stabilizer is at least one selected from the group consisting of neutral hydroxy amino acids, dipeptides of neutral hydroxy amino acids, tripeptides of neutral hydroxy amino acids, derivatives of neutral hydroxy amino acids and salts thereof.

5. The freeze-dried interferon-γ composition according to claim 1, wherein the hydrophilic stabilizer is at least one selected from the group consisting of arginine, lysine, histidine, threonine, dipeptide of these amino acids, tripeptides of these amino acids, derivatives of these amino acids and salts thereof.

6. The freeze-dried interferon-γ composition according to claim 1, wherein the hydrophobic stabilizer is at least one selected from the group consisting of hydrophobic amino acids, dipeptides of hydrophobic amino acids, tripeptides of hydrophobic amino acids, derivatives of hydrophobic amino acids and salts thereof.

7. The freeze-dried interferon-γ composition according to claim 1, wherein the hydrophobic stabilizer is at least one selected from the group consisting of valine, leucine, isoleucine, phenylalanine and salts thereof.

8. The freeze-dried interferon-γ composition according to claim 1, wherein the content of the hydrophilic stabilizer is 1 to 500 parts by weight per 100 parts by weight of the hydrophobic stabilizer.

9. The freeze-dried interferon-γ composition according to claim 1, wherein the disintegration index is 0.02 or more.

10. The freeze-dried interferon-γ composition according to claim 1, wherein the disintegration index is from 0.015 to 1.5.

11. The freeze-dried interferon-γ composition according to claim 1, becoming fine particles having a mean particle diameter of 10 microns or less or a fine particle fraction of 10% or more upon receipt of an air impact having an air speed of at least 2 m/sec and an air flow rate of at least 17 ml/sec.

12. The freeze-dried interferon-γ composition according to claim 1, becoming fine particles having a mean particle diameter of 10 microns or less or a fine particle fraction of 10% or more upon receipt of an air impact having an air speed of at least 1 m/sec and an air flow rate of at least 20 ml/sec.

13. The freeze-dried interferon-γ composition according to claim 1, becoming fine particles having a mean particle diameter of 5 microns or less or a fine particle fraction of 20% or more upon receipt of the air impact.

14. The freeze-dried interferon-γ composition for transpulmonary administration according to claim 1, having the following properties (i) to (iv):
(i) containing at least one hydrophobic stabilizer selected from the group consisting of hydrophobic amino acids, dipeptides of hydrophobic amino acids, tripeptides of hydrophobic amino acids, derivatives of hydrophobic amino acids and salts thereof; at least one hydrophilic stabilizer selected from the group consisting of hydrophilic amino acids, dipeptides of hydrophilic amino acids, tripeptides of hydrophilic amino acids, derivatives of hydrophilic amino acids and salts thereof; and interferon-γ;
(ii) a non-powder cake-like form;
(iii) a disintegration index of 0.015 to 1.5; and
(iv) becoming fine particles having a mean particle diameter of 10 microns or less or a fine particle fraction of 10% or more upon receipt of an air impact having an air speed in the range of 1 to 300 m/sec and an air flow rate in the range of 17 ml/sec to 15 L/sec.

15. A dry powder interferon-γ inhalation system for transpulmonary administration, using a combination of:
(1) a vessel housing the freeze-dried interferon-γ composition for transpulmonary administration according to claim 1; and
(2) a device comprising means capable of applying an air impact having an air speed of at least 1 m/sec and an air flow rate of at least 17 ml/sec to the freeze-dried composition in said vessel, and means for discharging the powder-form freeze-dried composition that has been made into fine particles.

16. The dry powder interferon-γ inhalation system for transpulmonary administration according to claim 15, wherein the vessel and the device are used in combination at the time of inhalation.

17. The dry powder interferon-γ inhalation system for transpulmonary administration according to claim 15, wherein the device is:
i) a dry powder inhaler for transpulmonary administration, being a device used for making a freeze-dried composition that has been housed in non-powder form in a vessel into fine particles, and administering the resulting fine particles to a user by inhalation,
comprising a needle part having an air jet flow path, a needle part having a discharge flow path, air pressure-feeding means for feeding air into the air jet flow path of said needle part, and an inhalation port that communicates with the discharge flow path of said needle part,
and characterized by being constituted such that a stopper that seals up said vessel is pierced by said needle parts, thus communicating the air jet flow path and the discharge flow path with the inside of said vessel, and air is jetted into said vessel through said air jet flow path using said air pressure-feeding means, thus making said freeze-dried composition into fine particles by the impact of the jetted air, and discharging the fine particles obtained from the inhalation port via said discharge flow path, or
ii) a dry powder inhaler for transpulmonary administration, being a device used for making a freeze-dried composition that has been housed in non-powder form in a vessel into fine particles, and administering the resulting fine particles to a user by inhalation,
comprising a needle part having a suction flow path, a needle part having an air introduction flow path, and an inhalation port that communicates with said suction flow path,
and characterized by being constituted such that, in a state in which a stopper sealing up said vessel has been pierced by said needle parts, through the inhalation pressure of the user, air in said vessel is inhaled from said inhalation port, and at the same time outside air flows into said vessel, at a negative pressure, through said air introduction flow path, and as a result said freeze-dried composition is made into fine particles by the impact of the air flowing in, and the fine particles obtained are discharged from the inhalation port through said suction flow path.

18. A dry powder interferon-γ inhalation system for transpulmonary administration, using a combination of:
(1) a vessel housing the freeze-dried interferon-γ composition for transpulmonary administration according to claim 14; and
(2) a device comprising means capable of applying said air impact to the freeze-dried composition in said vessel, and means for discharging the powder-form freeze-dried composition that has been made into fine particles.

19. A method of manufacturing a dry powdered interferon-γ preparation for transpulmonary administration, comprising:
introducing air into a vessel to apply to a freeze-dried composition containing a single dose of interferon-γ according to claim 1 an air impact having an air speed of at least 1 m/sec and an air flow rate of at least 17 ml/sec using a device capable of applying said air impact to the freeze-dried composition in the vessel, thereby making said freeze-dried composition into fine particles having a mean particle diameter of 10 microns or less or a fine particle fraction of 10% or more.

20. The method of manufacturing a dry powdered interferon-γ preparation for transpulmonary administration according to claim 19, wherein the fine particles prepared have a mean particle diameter of 5 microns or less or a fine particle fraction of 20% or more.

21. The method of manufacturing a dry powdered interferon-γ preparation for transpulmonary administration according to claim 19, carried out by using a device having means capable of applying an air impact having an air speed of at least 2 m/sec and an airflow rate of at least 17 ml/sec to the freeze-dried composition in the vessel, and introducing air having the air impact into the vessel housing the freeze-dried composition.

22. A method of manufacturing a dry powdered interferon-γ preparation for transpulmonary administration comprising:
introducing air into a vessel to apply to a freeze-dried composition containing a single dose of interferon-γ according to claim 14 an air impact having an air speed of 1 to 300 m/sec and an air flow rate of at least 17 ml/sec to 15 L/sec using a device capable of applying said air impact to the freeze-dried composition in the vessel,
thereby making said freeze-dried composition into fine particles having a mean particle diameter of 10 microns or less or a fine particle fraction of 10% or more.

23. A transpulmonary administration method comprising:
making the freeze-dried interferon-γ composition for transpulmonary administration containing a single dose of interferon-γ according to claim 1 into fine particles having a mean particle diameter of 10 microns or less or a fine particle fraction of 10% or more by applying an air impact having an air speed of at least 1 m/sec and an air flow rate of at least 17 ml/sec to the freeze-dried composition at the time of use, and
administering the resulting fine particle powder to a user by inhalation.

24. The transpulmonary administration method according to claim 23, wherein the freeze-dried interferon-γ composition for transpulmonary administration is housed in a vessel, and the fine particle powder are made using a device comprising means capable of applying the air impact to the freeze-dried composition in the vessel and means for discharging the resulting fine particle powder-form freeze-dried composition out of the vessel.

25-27. (canceled)
